(19) United States
(12) Patent Application Publication
(10) Pub. No.: US 2018/0080063 A1
(43) Pub. Date: Mar. 22, 2018

(54) APPARATUS AND METHODS FOR CONDUCTING CHEMICAL REACTIONS

(71) Applicant: Coyote Bioscience Co., Ltd., Beijing (CN)

(72) Inventors: Xiang Li, Beijing (CN); Xiaobing Mu, Beijing (CN); Chen Li, Hefei City (CN); Huiying Feng, Beijing (CN); Yang Zhou, Tongliao City (CN); Yuguang Han, Chengde City (CN); Juan Chen, Beijing (CN); Yachao Hu, Shijizhuang City (CN)

(21) Appl. No.: 15/609,877

(22) Filed: May 31, 2017

Related U.S. Application Data
(63) Continuation of application No. PCT/CN2015/100138, filed on Dec. 31, 2015.
Application No. PCT/CN2015/100138 is a continuation-in-part of application No. PCT/CN2015/074513, filed on Mar. 18, 2015.
Application No. PCT/CN2015/074513 is a continuation-in-part of application No. PCT/CN2014/095987, filed on Dec. 31, 2014.

(51) Int. Cl. C12Q 1/68; B01L 7/00

(52) U.S. Cl. CPC C12Q 1/686; B01L 7/5255; B01L 2300/1894; B01L 2300/18; B01L 2200/025

(57) ABSTRACT

Provided herein are devices, systems, and methods for conducting nucleic acid amplification. The devices, systems, and methods are suited for portability, rapid operation, low power consumption, integrated operation, and remote monitoring.

### CROSS-REFERENCE

This application is a continuation of PCT Application Ser. No. PCT/CN2015/100138, filed Dec. 31, 2015, which is a continuation-in-part of PCT Application Ser. No. PCT/CN2015/074513, filed Mar. 18, 2015 and is a continuation-in-part of PCT Application Serial No. PCT/CN2014/095987, filed Dec. 31, 2014, which applications are herein entirely incorporated by reference in their entireties for all purposes.

### BACKGROUND

Polymerase chain reaction (PCR) is a widely-used technique in molecular biology for the amplification of nucleic acid molecules. PCR relies on thermocycling, cycles of repeated heating and cooling of the reaction mixture. These cycles allow for melting and enzymatic replication of the nucleic acids.

In an appropriate reagent reaction system, the polymerase can perform the chain reaction at a rapid speed. In fact, amplification of nucleic acid molecules in a polymerase chain reaction (PCR) can occur in one to two seconds, or even less than one second per cycle. Therefore, in many situations, the speed of PCR amplification is limited by the performance of the instrumentation (e.g. thermal cycler) rather than the biological reaction itself.

### SUMMARY

Recognized herein is the need for improved systems and methods for thermocycling, for reactions such as PCR. Reducing the time for heating and cooling sample volumes between the necessary temperature set points can reduce the time required to conduct a reaction cycle and therefore reduce the overall reaction time over multiple cycles.

An aspect of the present disclosure provides a method of conducting a chemical reaction in a sample contained in a sample holder, the reaction requiring cycling between at least two target temperature levels, the method comprising: (a) placing the sample holder in thermal contact with a first overshooting thermal zone to achieve a first target temperature level; (b) placing the sample holder in thermal contact with a second overshooting thermal zone to achieve a second target temperature level; and in some cases repeating steps (a) and (b); wherein the first overshooting thermal zone is at a higher temperature than the first target temperature level, and the second overshooting thermal zone is at a lower temperature than the second target temperature level.

In some embodiments of aspects provided herein, the step of (a) referenced above is performed with the aid of a first rotating arm capable of placing the first overshooting thermal zone in thermal contact with the sample holder, and/or wherein step (b) is performed with the aid of a second rotating arm capable of placing the second overshooting thermal zone in thermal contact with the sample holder. In some embodiments of aspects provided herein, the first overshooting thermal zone is mounted on the first rotating arm, and wherein the second overshooting thermal zone is mounted on the second rotating arm. In some embodiments of aspects provided herein, the method further comprises: in between steps (a) and (b), the step of placing the sample holder in thermal contact with a first target thermal zone at the first target temperature level; and/or after step (b), placing the sample holder in thermal contact with a second target thermal zone at the second target temperature level. In some embodiments of aspects provided herein, the method further comprises: (c) in between steps (a) and (b), placing the sample holder in thermal contact with a first target thermal zone at the first target temperature level; and (d) after step (b), placing the sample holder in thermal contact with a second target thermal zone at the second target temperature level. In some embodiments of aspects provided herein, the first overshooting thermal zone is at a temperature from about 110° C. to about 140° C. In some embodiments of aspects provided herein, the first overshooting thermal zone is at a temperature of at least about 120° C. or 130° C. In some embodiments of aspects provided herein, the second overshooting thermal zone is at a temperature from about 0° C. to about 30° C. In some embodiments of aspects provided herein, the second overshooting thermal zone is at a temperature of less than or equal to about 8° C. In some embodiments of aspects provided herein, the first target temperature level is from about 87° C. to about 95° C. In some embodiments of aspects provided herein, the second target temperature level is from about 40° C. to about 70° C. In some embodiments of aspects provided herein, the second target temperature level is about 50° C. to about 55° C. In some embodiments of aspects provided herein, the first overshooting thermal zone is about 110° C. to 140° C., the first target temperature zone is about 87° C. to about 95° C., the second target temperature zone is about 40° C. to about 70° C., and the second overshooting thermal zone is about 0° C. to about 30° C. In some embodiments of aspects provided herein, one cycle of steps (a) through (d) is completed in less than or equal to about 2 seconds. In some embodiments of aspects provided herein, steps (a) through (d) are repeated at least 5 times. In some embodiments of aspects provided herein, the first overshooting thermal zone and the second overshooting thermal zone are powered by a 12 volt power supply. In some embodiments of aspects provided herein, the sample holder is pre-loaded with amplification reagent prior to collecting the sample in the sample holder.

In some embodiments of aspects provided herein, the sample holder is placed in thermal communication with the first overshooting thermal zone and the second overshooting thermal zone using a first translational unit and a second translational unit. The first translational unit may subject the first overshooting thermal zone and the second overshooting thermal zone to movement along a first plane, and the second translational unit may subject the sample holder to movement along a second plane that is angled with respect to the first plane. In some embodiments, operation (a) comprises using the first translational unit to move the first overshooting thermal zone to a first position and the second overshooting thermal zone to a second position along the first plane when the sample holder is raised away from the first plane, and subsequently using the second translational unit to lower the sample holder towards the first plane such that the sample holder is brought in thermal communication with the first overshooting thermal zone. In some embodiments, operation (b) comprises using the first translational unit to move the second overshooting thermal zone to the first position and the first overshooting thermal zone to a third position when the sample holder is raised away from the first plane, and subsequently use the second translational unit to lower the sample holder towards the first plane such that the sample holder is brought in thermal communication with the second overshooting thermal zone. The third position may be different than the second position. As an alternative, the third position may be the same as the second position. In some embodiments, the first translational unit subjects the first overshooting thermal zone and the second overshooting thermal zone to simultaneous movement along the first plane. The movement along the second plane may be towards or away from the first plane. The second plane may be at an angle from about 45° to 90° with respect to the first plane.

In some embodiments of aspects provided herein, the step of (a) referenced above is performed with the aid of a swinging arm capable of placing the sample holder in thermal contact with the first overshooting thermal zone, and/or wherein step (b) is performed with the aid of the swinging arm capable of placing the sample holder in thermal contact with the second overshooting thermal zone. In some embodiments of aspects provided herein, the sample holder is mounted on the swinging arm. In some embodiments of aspects provided herein, the method further comprises: in between steps (a) and (b), the step of switching alternately between making the sample holder in and out of thermal contact with the first overshooting thermal zone so that the sample holder is maintained at the first target temperature level; and/or after step (b), placing the sample holder in thermal contact again with the first overshooting thermal zone and switching alternately between making the sample holder in and out of thermal contact with the first overshooting thermal zone so that the sample holder is maintained at the second target temperature level. In some embodiments of aspects provided herein, the method further comprises: (c) in between steps (a) and (b), switching alternately between placing the sample holder in and out of thermal contact with the first overshooting thermal zone so that the sample holder is maintained at the first target temperature level; and (d) after step (b), placing the sample holder in thermal contact again with the first overshooting thermal zone and switching alternately between making the sample holder in and out of thermal contact with the first overshooting thermal zone so that the sample holder is maintained at the second target temperature level. In some embodiments of aspects provided herein, the first overshooting thermal zone is at a temperature from about 110° C. to about 140° C. In some embodiments of aspects provided herein, the first overshooting thermal zone is at a temperature of at least about 120° C. or 130° C. In some embodiments of aspects provided herein, the second overshooting thermal zone is at a temperature from about 0° C. to about 30° C. In some embodiments of aspects provided herein, the second overshooting thermal zone is at a temperature of less than or equal to about 8° C. In some embodiments of aspects provided herein, the first target temperature level is from about 87° C. to about 95° C. In some embodiments of aspects provided herein, the second target temperature level is from about 40° C. to about 70° C. In some embodiments of aspects provided herein, the second target temperature level is about 50° C. to about 55° C. In some embodiments of aspects provided herein, the first overshooting thermal zone is about 110° C. to 140° C., the first target temperature zone is about 87° C. to about 95° C., the second target temperature zone is about 40° C. to about 70° C., and the second overshooting thermal zone is about 0° C. to about 30° C. In some embodiments of aspects provided herein, one cycle of steps (a) through (d) is completed in less than or equal to about 2 seconds. In some embodiments of aspects provided herein, steps (a) through (d) are repeated at least 5 times. In some embodiments of aspects provided herein, the first overshooting thermal zone and the second overshooting thermal zone are powered by a 12 volt power supply. In some embodiments of aspects provided herein, the sample holder is pre-loaded with amplification reagent prior to collecting the sample in the sample holder.

An aspect of the present disclosure provides a method of conducting a chemical reaction in a sample, the reaction requiring cycling between at least two temperature levels, the method comprising: thermocycling the sample between a first target temperature level of about 87° C. to about 95° C. and a second target temperature level of about 40° C. to about 70° C.; wherein a time to complete a cycle of the thermocycling is less than or equal to about 5 seconds; and wherein the sample has a volume of at least about 1 microliter.

In some embodiments of aspects provided herein, the chemical reaction is a nucleic acid amplification reaction. In some embodiments of aspects provided herein, the chemical reaction is a PCR reaction. In some embodiments of aspects provided herein, the first target temperature level is about 50° C. to about 55° C. In some embodiments of aspects provided herein, the time is less than or equal to about 2 seconds. In some embodiments of aspects provided herein, the time is less than or equal to about 1 second. In some embodiments of aspects provided herein, the time is less than or equal to about 0.5 seconds. In some embodiments of aspects provided herein, the volume is at least about 5 microliters. In some embodiments of aspects provided herein, the volume is at least about 10 microliters. In some embodiments of aspects provided herein, the volume is at least about 20 microliters. In some embodiments of aspects provided herein, the volume is at least about 50 microliters. In some embodiments of aspects provided herein, the volume is at least about 100 microliters. In some embodiments of aspects provided herein, the volume is at least about 150 microliters. In some embodiments of aspects provided herein, the volume is at least about 200 microliters.

An aspect of the present disclosure provides an apparatus for conducting a chemical reaction in a sample, the reaction requiring cycling between at least two target temperature levels, comprising: (a) a first overshooting thermal zone which when in operation is maintained at about 110° C. to about 140° C.; (b) a first target thermal zone which when in operation is maintained at from about 92° C. to about 95° C.; (c) a second overshooting thermal zone which when in operation is maintained at about 0° C. to about 30° C.; (d) a second target thermal zone which when in operation is maintained at about 40° C. to about 70° C.; (e) a sample holder configured to hold one or more samples; and (f) one or more arms programmed to place the sample holder in thermal contact with one or more of zones of (a) through (d) in a sequence.

In some embodiments of aspects provided herein, the one or more arms comprises a target thermal zone or an overshooting thermal zone. In some embodiments of aspects provided herein, (a) the first overshooting thermal zone is mounted on a first rotating arm capable of placing the first overshooting thermal zone into thermal contact with the sample holder, (b) the first target thermal zone is mounted on a second rotating arm capable of placing the first target thermal zone into thermal contact with the sample holder, (c) the second overshooting thermal zone is mounted on a third rotating arm capable of placing the second overshooting thermal zone into thermal contact with the sample holder, and (d) the second target thermal zone is mounted on a fourth rotating arm capable of placing the second target thermal zone into thermal contact with the sample holder. In some embodiments of aspects provided herein, the first overshooting thermal zone when in operation is maintained at a temperature of at least about 120° C. or 130° C. In some embodiments of aspects provided herein, the first target thermal zone when in operation is maintained at greater than or equal to about 95° C. In some embodiments of aspects provided herein, the second overshooting thermal zone when in operation is maintained at less than or equal to about 8° C. In some embodiments of aspects provided herein, the second target thermal zone when in operation is maintained at about 50° C. to about 55° C. In some embodiments of aspects provided herein, the device further comprises an optical module comprising an optical detector. In some embodiments of aspects provided herein, the first overshooting thermal zone, the first target thermal zone, the second overshooting thermal zone, and the second target thermal zone are powered by a 12 volt power supply.

An aspect of the present disclosure provides an apparatus for conducting a chemical reaction in a sample, the reaction requiring cycling between at least two target temperature levels, comprising: (a) a first overshooting thermal zone which when in operation is maintained at about 110° C. to about 140° C.; (b) a second overshooting thermal zone which when in operation is maintained at about 0° C. to about 35° C.; (c) a sample holder configured to hold one or more samples; and (d) one or more swinging arms programmed to place the sample holder in thermal contact with the zones of (a) and (b) sequentially.

In some embodiments of aspects provided herein, the one or more swinging arm comprises the sample holder. In some embodiments of aspects provided herein, the sample holder is mounted on the one of more swinging arms capable of placing the sample holder into thermal contact with the first and/or second overshooting thermal zone. In some embodiments of aspects provided herein, the first overshooting thermal zone and/or the sample holder is configured so that it is possible for them to switch between the state of in and out of thermal contact with each other, thereby maintaining the sample holder at a first target temperature level and/or a second target temperature level. In some embodiments of aspects provided herein, the first overshooting thermal zone comprises a first heating module and a second heating module, which are configured to switch between an open position and a closed position, wherein in the open position, none of the heating modules is in thermal contact with the sample holder, while in the closed position, at least one (and preferably both) of the first and the second heating module is in thermal contact with the sample holder.

In some embodiments of aspects provided herein, the first overshooting thermal zone when in operation is maintained at greater than or equal to about 120° C. or 130° C. In some embodiments of aspects provided herein, the first target temperature level is maintained at greater than or equal to about 95° C. In some embodiments of aspects provided herein, the second overshooting thermal zone when in operation is maintained at less than or equal to about 8° C. In some embodiments of aspects provided herein, the second target temperature level is maintained at about 50° C. to about 55° C. In some embodiments of aspects provided herein, the apparatus further comprises an optical module comprising an optical detector. In some embodiments of aspects provided herein, the first overshooting thermal zone and the second overshooting thermal zone are powered by a 12 volt power supply. In some embodiments of aspects provided herein, thermal insulation material may be provided between the first overshooting thermal zone and the second overshooting thermal zone, so as to avoid conduction of heat.

Another aspect of the present disclosure provides a system for amplifying a target nucleic acid present in a biological sample obtained from a subject. The system may comprise an input module that receives a request from a user to amplify the target nucleic acid in the biological sample. The system may further comprise an amplification module that, in response to the user request: (i) receives, in a reaction vessel held by a sample holder, a reaction mixture comprising the biological sample and reagents necessary for conducting nucleic acid amplification, the reagents comprising (i) a DNA polymerase and optionally a reverse transcriptase, and (ii) a primer set for the target nucleic acid; and (ii) subjects the reaction mixture in the reaction vessel to a plurality of series of primer extension reactions to generate amplified product(s) that is indicative of a presence of the target nucleic acid in the biological sample. Each series may comprise cycling between at least two target temperature levels for one or more cycles of: (a) placing the sample holder in thermal communication with a first overshooting thermal zone to achieve a first target temperature level; and (b) placing the sample holder in thermal communication with a second overshooting thermal zone to achieve a second target temperature level, wherein the first overshooting thermal zone is at a higher temperature than the first target temperature level, and the second overshooting thermal zone is at a lower temperature than the second target temperature level. The system may further comprise an output module operatively coupled to the amplification module. The output module may output information related to the target nucleic acid or the amplified product(s) to a recipient.

In some embodiments, the system may further comprise an identification unit that uniquely identifies the system. The identification unit is detectable by an electronic device of the user, wherein during use, (i) the identification unit is detected by the electronic device to identify the system, and (ii) upon the system being identified, the request is directed from the electronic device to the system. The identification unit may be an identification number or barcode. In some embodiments, the identification unit is a radiofrequency identification (RFID) unit.

An aspect of the present disclosure provides a system for amplifying a target nucleic acid in a biological sample obtained from a subject. The system may comprise an electronic display screen comprising a user interface that displays a graphical element that is accessible by a user to execute an amplification protocol to amplify the target nucleic acid in the biological sample; and one or more computer processors coupled to the electronic display screen and individually or collectively programmed to execute the amplification protocol upon selection of the graphical element by the user. The amplification protocol may comprise subjecting a reaction mixture comprising the biological sample and reagents necessary for conducting nucleic acid amplification contained in a reaction vessel held by a sample holder to a plurality of series of primer extension reactions to generate amplified product(s) that is indicative of a presence of the target nucleic acid in the biological sample. Each series may comprise cycling between at least two target temperature levels for one or more cycles of : (a) placing the sample holder in thermal communication with a first overshooting thermal zone to achieve a first target temperature level; and (b) placing the sample holder in thermal communication with a second overshooting thermal zone to achieve a second target temperature level, wherein the first overshooting thermal zone is at a higher temperature than the first target temperature level, and wherein the second overshooting thermal zone is at a lower temperature than the second target temperature level. The amplification protocol may further comprise selecting a primer set for the target nucleic acid. The reagents may comprise (i) a deoxyribonucleic acid (DNA) polymerase and optionally a reverse transcriptase, and (ii) a primer set for the target nucleic acid. The user interface may display a plurality of graphical elements, wherein each of the graphical elements is associated with a given amplification protocol among a plurality of amplification protocols.

Each of the graphical elements may be associated with a disease or health condition, and a given amplification protocol among the plurality of amplification protocols may be directed to assaying a presence of the disease or health condition in the subject. The disease or health condition may be associated with a single nucleotide polymorphism (SNP) or with a virus, the virus may be an RNA virus and/or a DNA virus. In some embodiments, the virus may be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, FluA virus, respiratory syncytial virus A (RSVA), respiratory syncytial virus B (RSVB), measles virus, Varicella virus, H1N1 virus, H3N2 virus, H7N9 virus, H5N1 virus, adenovirus type 55 (ADV55), adenovirus type 7 (ADV7), and armored RNA-hepatitis C virus (RNA-HCV).

In some embodiments, the disease or health condition is associated with a pathogenic bacterium or a pathogenic protozoan. The pathogenic bacterium may be Mycobacterium tuberculosis. The pathogenic protozoan may be Plasmodium.

The system may further comprise an identification unit that uniquely identifies the system. The identification unit is detectable by an electronic device of the user, wherein during use, (i) the identification unit is detected by the electronic device to identify the system, and (ii) upon the system being identified, a request to execute the amplification is directed from the electronic device to the system. The identification unit may be an identification number or barcode. In some embodiments, the identification unit is a radiofrequency identification (RFID) unit.

Another aspect of the present disclosure provides an apparatus for conducting a reaction on a sample. The apparatus may comprise a sample holder that holds the sample during the reaction, wherein the reaction comprises cycling between at least two target temperature levels including a first target temperature level and a second target temperature level. The apparatus may further comprise a first overshooting thermal zone and a second overshooting thermal zone, wherein the first overshooting thermal zone is at a higher temperature than the first target temperature level and the second overshooting thermal zone is at a lower temperature than the second target temperature level, or vice versa. The apparatus may also comprise a controller that is programmed to alternately and sequentially (i) place the sample holder in thermal communication with the first overshooting thermal zone to achieve the first target temperature level, and (ii) place the sample holder in thermal communication with the second overshooting thermal zone to achieve the second target temperature level.

In some embodiments, the apparatus further comprises a first translational unit and a second translational unit. The first translational unit may subject the first overshooting thermal zone and the second overshooting thermal zone to movement along a first plane, and the second translational unit may subject the sample holder to movement along a second plane that is angled with respect to the first plane. The controller may be operatively coupled to the first translational unit and the second translational unit, and the controller may be programmed to subject the first overshooting thermal zone and the second overshooting thermal zone to movement along the first plane, and subject the sample holder to movement along the second plane, to alternately and sequentially (i) place the sample holder in thermal communication with the first overshooting thermal zone to achieve the first target temperature level, and (ii) place the sample holder in thermal communication with the second overshooting thermal zone to achieve the second target temperature level. The first translational unit may subject the first overshooting thermal zone and the second overshooting thermal zone to simultaneous movement along the first plane. The movement along the second plane may be towards or away from the first plane. The second plane may be at an angle from about 45° to 90° with respect to the first plane.

In some embodiments, the controller is programmed to: (1) direct the first translational unit to move the first overshooting thermal zone to a first position and the second overshooting thermal zone to a second position along the first plane when the sample holder is raised away from the first plane; (2) direct the second translational unit to lower the sample holder towards the first plane, thereby placing the sample holder in thermal communication with the first overshooting thermal zone to achieve the first target temperature level; (3) direct the first translational unit to move the second overshooting thermal zone to the first position and the first overshooting thermal zone to a third position when the sample holder is raised away from the first plane; and (4) direct the second translational unit to lower the sample holder towards the first plane, thereby placing the sample holder in thermal communication with the second overshooting thermal zone to achieve the second target temperature level. The third position may be different than the second position. As an alternative, the third position may be the same as the second position. The controller may be programmed to direct the second translational unit to raise the sample holder away from the first plane between (2) and (3). The first translational unit and/or the second translational unit may include at least one motor or piezoelectric actuator. The first translational unit and/or the second translational unit may include a track. The track may be a linear track.

The first overshooting thermal zone may be at a temperature from about 110° C. to about 140° C. In some embodiments, the first overshooting thermal zone is at a temperature of at least about 120° C. or 130° C. In some embodiments, the first overshooting thermal zone is a heating unit. The second overshooting thermal zone may be at a temperature from about 0° C. to about 30° C. In some embodiments, the second overshooting thermal zone is a cooling unit. The sample holder may hold a plurality of samples.

Other goals and advantages of the invention will be further appreciated and understood when considered in conjunction with the following description and accompanying drawings. While the following description may contain specific details describing particular embodiments of the invention, this should not be construed as limitations to the scope of the invention but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows an exemplary schematic of a thermocycler device.

FIG. 1B shows an exemplary schematic of a thermocycler device with a sample holder in thermal contact with a first overshooting thermal zone.

FIG. 1C shows an exemplary schematic of a thermocycler device with a sample holder in thermal contact with a second overshooting thermal zone.

FIG. 2 shows an exemplary graph of temperature over time for a PCR reaction.

FIG. 3 shows an exemplary three-quarter view schematic of a thermocycler device.

FIG. 4 shows an exemplary exploded three-quarter view schematic of a thermocycler device.

FIG. 5 shows an exemplary side view schematic of a thermocycler device.

FIG. 6A shows an exemplary side view schematic of a thermocycler device with a sample holder in thermal contact with a first overshooting thermal zone.

FIG. 6B shows an exemplary side view schematic of a thermocycler device with a sample holder in thermal contact with a first target thermal zone.

FIG. 6C shows an exemplary side view schematic of a thermocycler device with a sample holder in thermal contact with a second overshooting thermal zone.

FIG. 6D shows an exemplary side view schematic of a thermocycler device with a sample holder in thermal contact with a second target thermal zone.

FIG. 7 shows an exemplary schematic of a thermocycler device.

FIG. 8 shows an exemplary schematic of a thermocycler device.

FIG. 9 shows an exemplary computer control system that is programmed or otherwise configured to implement methods provided herein.

FIG. 10 (panel A) shows an exemplary top view of the external enclosure of a thermocycler device.

FIG. 10 (panel B) shows an exemplary bottom view of the external enclosure of a thermocycler device.

FIG. 10 (panel C) shows an exemplary stereo view of the external appearance of a thermocycler device.

FIG. 10 (panel D) shows an exemplary enlarged partial view of the top of a thermocycler device with the lid open.

FIG. 11 shows an exemplary three-quarter view schematic of a thermocycler device, demonstrating its internal structures.

FIG. 12 shows an exemplary three-quarter view schematic of a thermocycler device, demonstrating its internal structures with a cover plate.

FIG. 13 shows an exemplary three-quarter view schematic of a thermocycler device, demonstrating its internal structures from the bottom.

FIG. 14 shows an exemplary three-quarter view schematic of a thermocycler device, demonstrating its internal structures from the bottom.

FIG. 15 shows an exemplary exploded three-quarter view schematic of a thermocycler device.

FIG. 16 (panel A) shows an exemplary three-quarter view schematic of a thermocycler with a sample holder in thermal contact with a second overshooting thermal zone.

FIG. 16 (panel B) shows an exemplary three-quarter view schematic of a thermocycler with a sample holder out of thermal contact with a second overshooting thermal zone.

FIG. 16 (panel C) shows an exemplary three-quarter view schematic of a thermocycler with a sample holder moved to the region of but not yet in thermal contact with a first overshooting thermal zone.

FIG. 16 (panel D) shows an exemplary three-quarter view schematic of a thermocycler with a sample holder in thermal contact with a first overshooting thermal zone.

FIG. 17 (panel A) shows an exemplary control panel of a thermocycler with an example electronic display having an example user interface.

FIG. 17 (panel B) shows an exemplary enlarged view of an example electronic display having an example user interface.

FIG. 18 shows an example user interface.

FIG. 19 shows an example user interface.

FIG. 20 shows an example user interface.

FIG. 21 shows an example user interface for running an experiment.

FIG. 22 (panel A) is a graph depicting results of nucleic acid amplification reactions. Curve A demonstrates results obtained for a target and curve B demonstrates results obtained for a control.

FIG. 22 (panel B) is a graph depicting results of nucleic acid amplification reactions. Curve A demonstrates results obtained for a target and curve B demonstrates results obtained for a control.

FIG. 22 (panel C) is a graph depicting results of nucleic acid amplification reactions. Curve A demonstrates results obtained for a target and curve B demonstrates results obtained for a control.

FIG. 23 shows an exemplary thermocycler device of the present disclosure. FIG. 23 (panel A) is a perspective side view, of the thermocycler, FIG. 23 (panel B) and FIG. 23 (panel C) show a top view and a bottom view, respectively, of the thermocycler. FIG. 23 (panel D) and FIG. 23 (panel E) show a front view and a back view, respectively, of the thermocycler. FIG. 23 (panel F) and FIG. 23 (panel G) show a left side view and a right side view, respectively, of the thermocycler.

FIG. 24 shows an exemplary three-quarter view schematic of a thermocycler device, demonstrating its internal structures.

FIG. 25 shows a partial internal structure of a thermocycler device of the present disclosure.

FIG. 26 shows a schematic perspective side view of a thermocycler device in operation. FIG. 26 (panels A-F) show various stages of operation of the thermocycler device.

FIG. 27 shows an example user interface.

FIG. 28 shows a graph depicting results of nucleic acid amplification reactions. Curve 1 demonstrates results obtained for hepatitis B virus and curve 2 demonstrates results obtained for a control.

FIG. 29 (panel A) and FIG. 29 (panel B) show a graph depicting results of nucleic acid amplification reactions. Curve 1 demonstrates results obtained for hepatitis C virus and curve 2 demonstrates results obtained for a control.

FIG. 30 shows a graph depicting results of nucleic acid amplification reactions for CYPC2C19.

### DETAILED DESCRIPTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The term “about” or “nearly” as used herein refers to within +/− 10%, 9%, 8%, 7%, 6%, 5%, 4%, 3%, 2%, or 1% of the designated amount.

The term “overshooting,” as used herein, generally refers to a point or region that is above or below a target or designated point or region. In some examples, in heating, an overshooting thermal zone may be at a temperature that is above a target temperature, and in cooling, an overshooting thermal zone may be at a temperature that is below a target temperature. For example, in heating a solution to 100° C., an overshooting thermal zone that is at a temperature of 140° C. is used. In another example, in cooling a solution to 25° C., an overshooting thermal zone that is at a temperature of 0° C. is used. An overshooting thermal zone may provide a greater temperature drop or temperature change, which may in turn provide a greater rate of heat transfer to provide heating or cooling, as necessary or required.

The term “thermal communication,” as used herein, generally refers to a state in which two or more materials are capable of exchange energy, such as thermal energy, with one another. Such exchange of energy may be by way of transfer of energy from one material to another material. Such transfer of energy may be radiative, conductive, or convective heat transfer. The energy may be thermal energy. In some examples, two or more materials that are in thermal communication with one another are in thermal contact with one another, such as, for example, direct physical contact or contact through one or more intermediary materials.

An aspect of the present disclosure provides a method of conducting a chemical reaction in a sample contained in a sample holder, the reaction requiring cycling between at least two target temperature levels, comprising: (a) placing the sample holder in thermal communication (e.g., thermal contact) with a first overshooting thermal zone to achieve a first target temperature level; (b) placing the sample holder in thermal communication with a second overshooting thermal zone to achieve a second target temperature level; and in some cases repeating steps (a) and (b); wherein the first overshooting thermal zone is at a higher temperature than the first target temperature level, and the second overshooting thermal zone is at a lower temperature than the second target temperature level.

Another aspect of the present disclosure provides a method of conducting a chemical reaction in a sample, the reaction requiring cycling between at least two temperature levels, the method comprising: thermocycling the sample between a first target temperature level of about 87° C. to about 95° C. and a second target temperature level of about 40° C. to about 70° C.; wherein a time to complete a cycle of the thermocycling is less than or equal to about 5 seconds; and wherein the sample has a volume of at least about 1 microliter.

The methods of the present disclosure can be used in conducting reactions or processes, such as polymerase chain reaction (PCR) amplifications of nucleic acids, which require cycling between two or more target temperature levels. Such thermocycling of a sample volume can be accomplished by (a) holding a thermal zone at a constant temperature and (b) thermally contacting a sample volume with the thermal zone to heat or cool the sample volume to the desired temperature (e.g., a set point temperature for a reaction). The rate of temperature change of the sample volume can be increased by contacting the sample volume with a thermal zone held at a temperature beyond the target temperature level; such a thermal zone can be referred to as an overshooting thermal zone. An overshooting thermal zone can be hotter than the target temperature level when heating a sample volume (e.g. the heating overshooting thermal zone). An overshooting thermal zone can be colder than the target temperature level when cooling a sample volume (e.g. the cooling overshooting thermal zone). If a protocol (e.g., for a reaction or process) calls for holding a sample volume at the target temperature level for a length of time, the sample volume can be contacted with an overshooting thermal zone for the heating or cooling step, and then contacted with a thermal zone held at the target temperature level for the holding step. Alternatively, in some embodiments of aspects provided herein, if a protocol (e.g., for a reaction or process) calls for holding a sample volume at the target temperature level for a length of time, the sample volume can be contacted with an overshooting thermal zone for the heating or cooling step, and then be placed again in thermal contact with the heating overshooting thermal zone and made to switch between the state of in and out of thermal contact with the heating overshooting thermal zone for the holding step.

For example, a PCR process may involve target temperature levels of 95° C. and 55° C. A PCR sample volume can be thermally contacted with a first overshooting thermal zone held constantly at 135° C. for rapid heating to 95° C., and then thermally contacted with a second overshooting thermal zone held constantly at 8° C. for rapid cooling to 55° C. If the process calls for holding the sample at the 95° C. target temperature level for a time, the sample can be rapidly heated to 95° C. with the first overshooting thermal zone, and then contacted with a first target thermal zone held constantly at 95° C. Similarly, if the process calls for holding the sample at the 55° C. target temperature level for a time, the sample can be rapidly cooled to 55° C. with the second overshooting thermal zone, and then contacted with a second target thermal zone held constantly at 55° C.

In another example, a PCR process can involve target temperature levels of 95° C. and 55° C. A PCR sample volume can be thermally contacted with a first overshooting thermal zone held constantly at 135° C. for rapid heating to 95° C., and then thermally contacted with a second overshooting thermal zone held constantly at 8° C. for rapid cooling to 55° C. If the process calls for holding the sample at the 95° C. target temperature level for a time, the sample can be rapidly heated to 95° C. with the first overshooting thermal zone, and then by making the sample to quickly switch between the state of in and out of thermal contact with the first overshooting thermal zone, the sample can be held constantly at 95° C. Similarly, if the process calls for holding the sample at the 55° C. target temperature level for a time, the sample can be rapidly cooled to 55° C. with the second overshooting thermal zone, and then by placing the sample into thermal contact again with the first overshooting thermal zone and making it to quickly switch between the state of in and out of thermal contact with the first overshooting thermal zone, the sample can be held constantly at 55° C.

In some embodiments, the sample holder may be placed in thermal communication with the first overshooting thermal zone and the second overshooting thermal zone using a first translational unit and a second translational unit. The first translational unit may subject the first overshooting thermal zone and the second overshooting thermal zone to movement (e.g., translational motion) along a first plane, and the second translational unit may subject the sample holder to movement along a second plane that is angled with respect to the first plane. Placing the sample holder in thermal communication (e.g., thermal contact) with a first overshooting thermal zone may comprise using the first translational unit to move the first overshooting thermal zone to a first position and the second overshooting thermal zone to a second position along the first plane when the sample holder is raised away from the first plane, and subsequently using the second translational unit to lower the sample holder towards the first plane such that the sample holder is brought in thermal communication with the first overshooting thermal zone. Placing the sample holder in thermal communication with a second overshooting thermal zone may comprise using the first translational unit to move the second overshooting thermal zone to the first position and the first overshooting thermal zone to a third position when the sample holder is raised away from the first plane, and subsequently use the second translational unit to lower the sample holder towards the first plane such that the sample holder is brought in thermal communication with the second overshooting thermal zone. The third position may be different than the second position. As an alternative, the third position may be the same as the second position. The first translational unit may subject the first overshooting thermal zone and the second overshooting thermal zone to simultaneous movement along the first plane. The movement along the second plane may be towards or away from the first plane. The second plane may be at an angle from about 45° to 90° with respect to the first plane.

The first position, second position and third position may be discrete positions. As an alternative, the first position, second position and third position may be semi-continuous positions, which may be bounded. The first position, second position and third position may be adjustable. In some cases, additional positions may be provided, such at least 1, 2, 3, 4, 5, 6, 7, 8, 9, 10, 20, 30, 40, or 50 additional positions. Each of the additional positions may correspond to a given configuration of an overshooting thermal zone.

An aspect of the present disclosure provides an apparatus for conducting a chemical reaction in a sample, the reaction requiring cycling between at least two target temperature levels, comprising: (a) a first overshooting thermal zone which when in operation is maintained at about 110° C. to about 140° C.; (b) a first target thermal zone which when in operation is maintained at from about 92° C. to about 95° C.; (c) a second overshooting thermal zone which when in operation is maintained at about 0° C. to about 30° C.; (d) a second target thermal zone which when in operation is maintained at about 40° C. to about 70° C.; (e) sample holder configured to hold one or more samples; and (f) one or more arms programmed to place the sample holder in thermal contact with one or more of zones of (a) through (d) in a sequence.

An aspect of the present disclosure provides an apparatus for conducting a chemical reaction in a sample, the reaction requiring cycling between at least two target temperature levels, comprising: (a) a first overshooting thermal zone which when in operation is maintained at about 110° C. to about 140° C.; (b) a second overshooting thermal zone which when in operation is maintained at about 0° C. to about 35° C.; (c) a sample holder configured to hold one or more samples; and (d) one or more swinging arms programmed to place the sample holder in thermal contact with the zones of (a) and (b) sequentially.

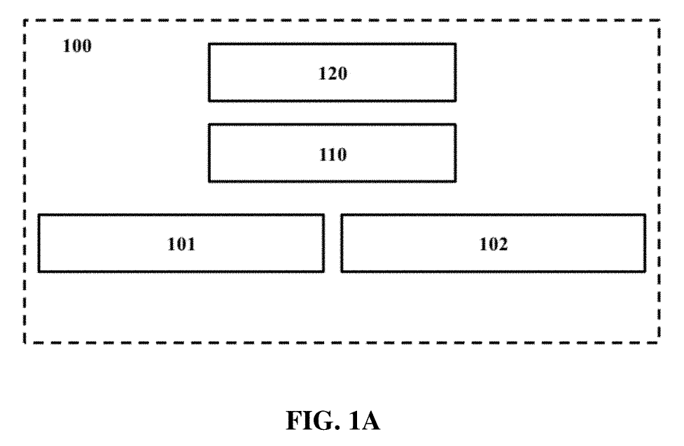

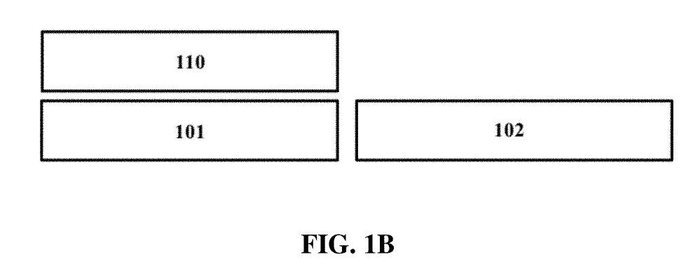

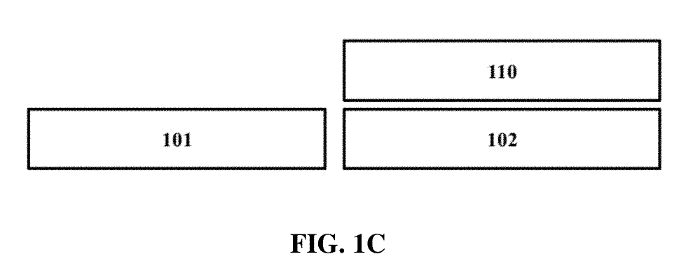

An apparatus of the present disclosure can be employed as a thermal cycler (herein also called thermocycler) for conducting reactions or processes, such as polymerase chain reaction (PCR) amplifications of nucleic acids, which require cycling between two or more target temperature levels. FIG. 1A shows a schematic of an exemplary thermal cycler device 100 comprising a first thermal overshoot zone 101, a second thermal overshoot zone 102, and a sample holder 110 which can hold one or more sample volumes. The device can also comprise a detector 120. The detector can be used to detect signal from the sample holder. FIG. 1B shows the sample holder 110 in thermal contact with the first thermal overshoot zone 101, and FIG. 1C shows the sample holder 110 in thermal contact with the second thermal overshoot zone 102.

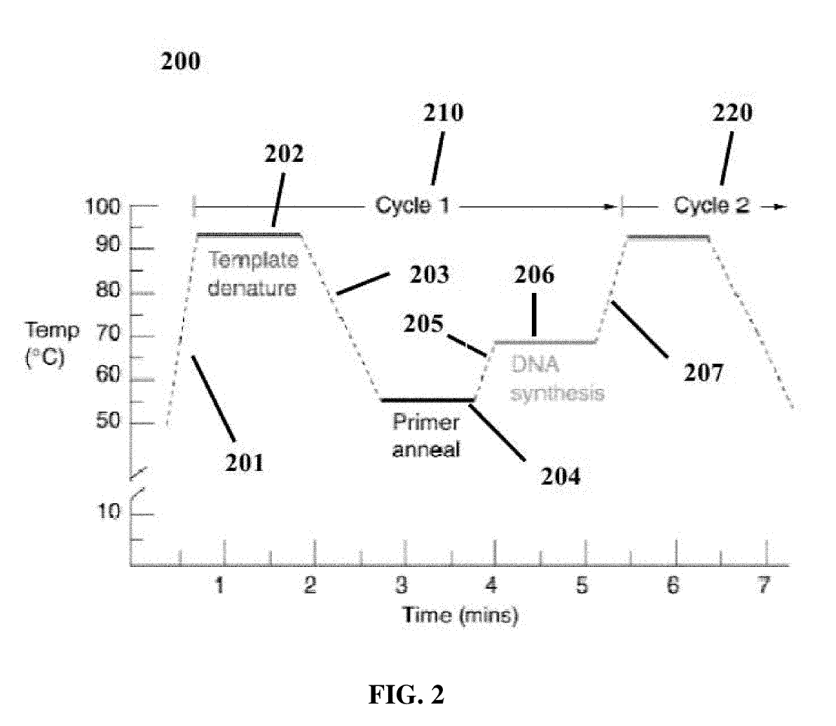

FIG. 2 shows an exemplary graph 200 of temperature over time for a PCR reaction thermal cycle. During the first temperature increase 201, the sample volume can be contacted with a first overshooting thermal zone for rapid heating. During the template denaturing step 202, the sample volume can be contacted with a first target thermal zone to maintain the first target temperature level. Alternatively, during the template denaturing step 202, the sample volume can be made to switch between the state of in and out of thermal contact with the first overshooting thermal zone to maintain the first target temperature level. During the first temperature decrease 203, the sample volume can be contacted with a second overshooting thermal zone for rapid cooling. During the primer annealing step 204, the sample volume can be contacted with a second target thermal zone to maintain the second target temperature level. Alternatively, during the primer annealing step 204, the sample volume can be placed into thermal contact again with the first overshooting thermal zone and made to switch between the state of in and out of thermal contact with the first overshooting thermal zone to maintain the second target temperature level. Additional thermal changes can be made, such as a second temperature increase 205 to a third target temperature level 206 (e.g., for DNA synthesis). The temperature can then be increased 207 back to the first target temperature level. The process of the first cycle 210 can be repeated for a second cycle 220 and as many subsequent cycles as are desired.

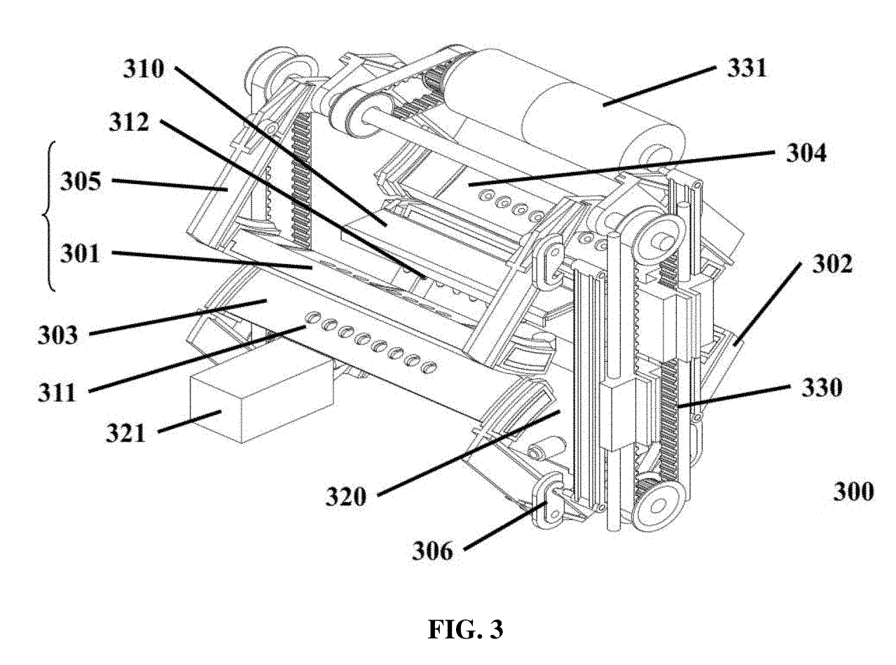

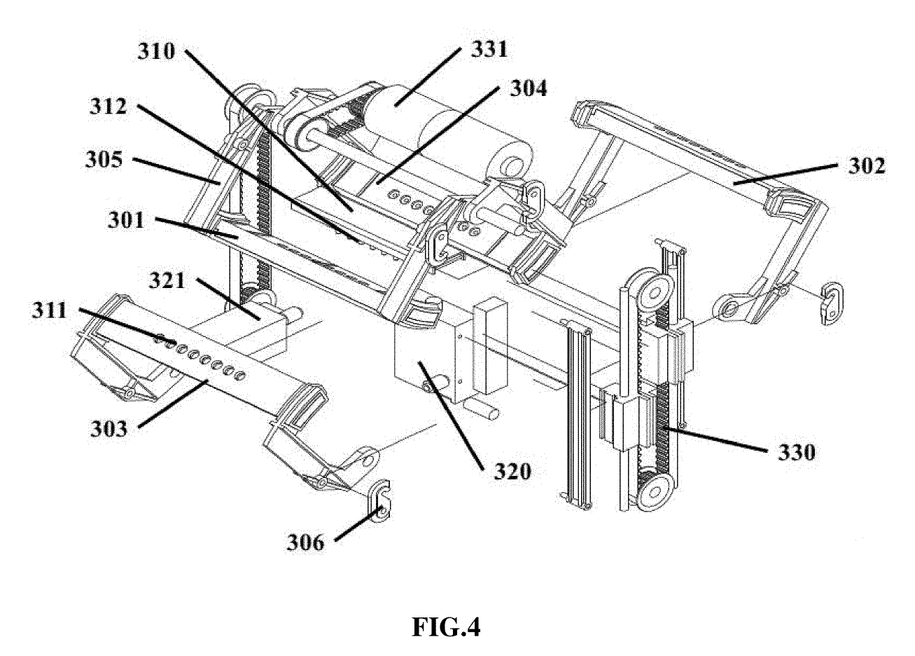

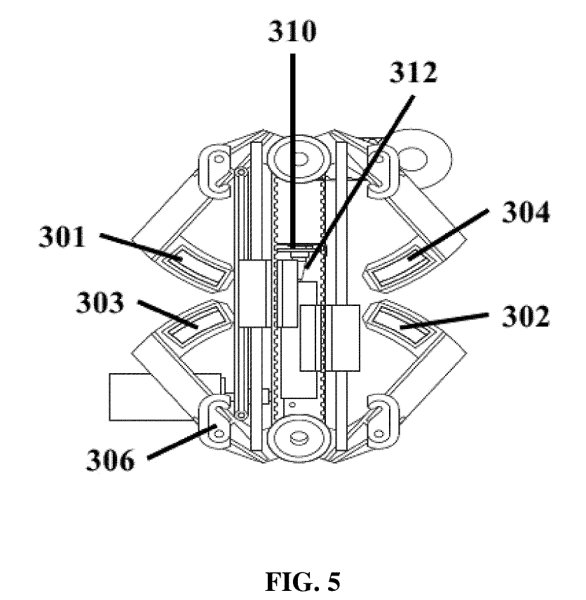

In another example, FIG. 3 shows a schematic of a thermocycler 300. This exemplary thermocycler comprises a first overshooting thermal zone 301 mounted on a first rotating arm 305, a second overshooting thermal zone 302 mounted on a second rotating arm, a first target thermal zone 303 mounted on a third rotating arm, and a second target thermal zone 304 mounted on a fourth rotating arm. The rotating arms can each comprise a hook 306 and be connected to a synchronous belt 330. The synchronous belt can be driven by a synchronous belt drive motor 331, and can also drive a heat lid with a sample holder 310 which can hold one or more reaction vessels 312 (e.g., PCR tubes, capillary tubes). The thermal zones can comprise tube wells 311 into which reaction vessels can fit for improved thermal contact. The thermocycler can also comprise an optical module 320 with a detector, and the optical module can be driven by an optical module drive motor 321. FIG. 4 shows an exploded schematic view of the exemplary thermocycler, and FIG. 5 shows a side view schematic of the exemplary thermocycler.

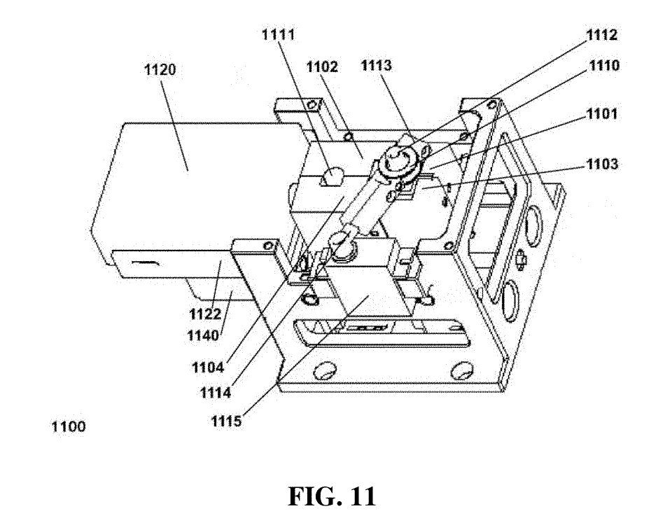

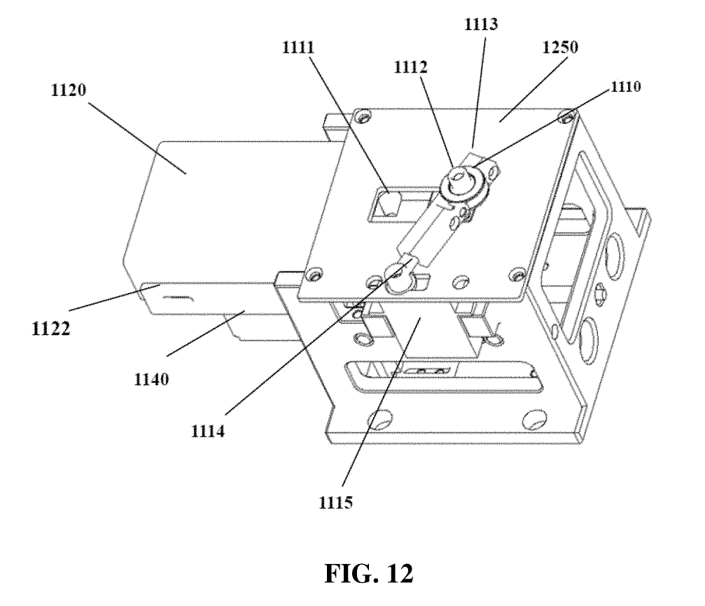

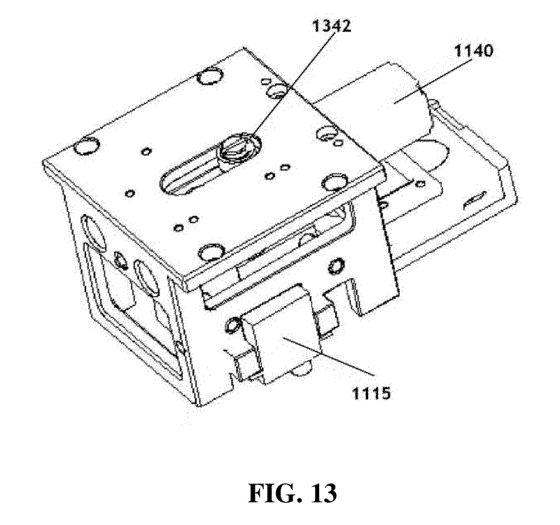

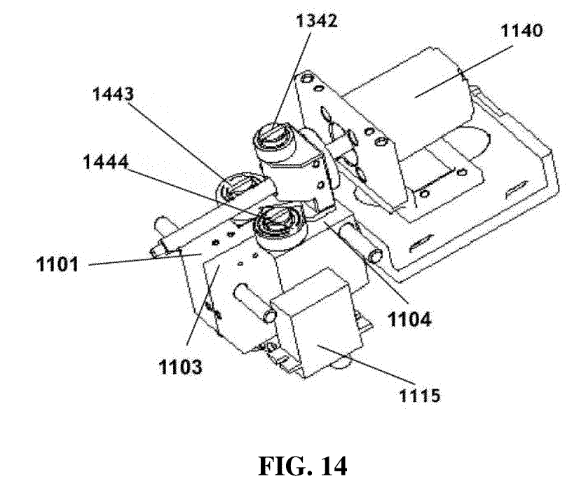

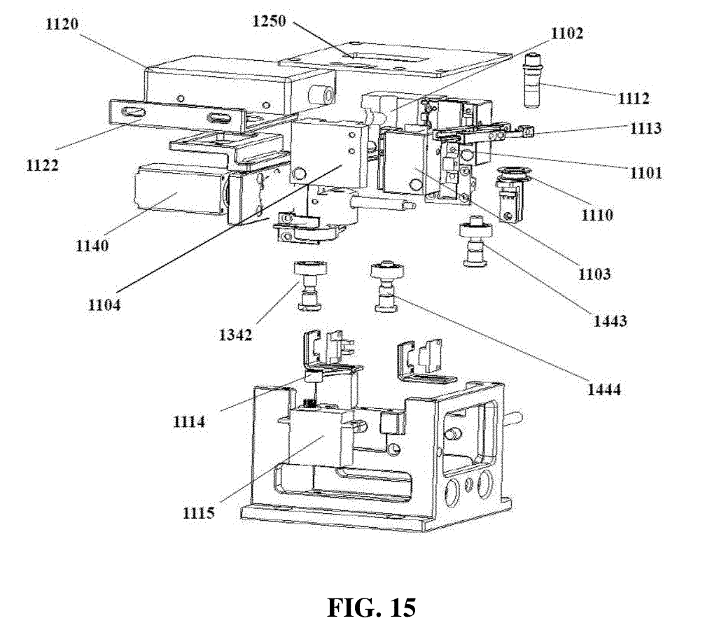

In another example, FIG. 11 shows a schematic of a thermocycler 1100. This exemplary thermocycler comprises: a first overshooting thermal zone composed of a first heating module 1101 and a second heating module 1103; a second overshooting thermal zone composed of a first cooling module 1102 and a second cooling module 1104; a sample holder 1110 mounted on a swinging arm 1114 capable of holding one or more reaction vessels 1112 (e.g., PCR tubes, capillary tubes). A sample rack 1113 may be further mounted on the swinging arm 1114 and the sample holder 1110 can be inserted in the sample rack 1113. The thermal zones can comprise tube wells 1111 into which reaction vessels can fit for improved thermal contact. The thermocycler can also comprise an optical module 1120 with a detector, and the optical module 1120 can be mounted on an optical module holder 1122. The swinging arm 1114 can be driven by an engine (e.g. a steering engine) 1115. The thermocycler 1100 can further comprise a motor (e.g. a stepper motor) 1140 to drive the heating modules 1101 and 1103 and/or the cooling modules 1102 and 1104 to switch between an open position and a closed position, wherein in the closed position the reaction vessel 1112 will be in thermal contact with the heating modules 1101 and 1103 or the cooling modules 1102 and 1104, and in the open position the reaction vessel 1112 will be out of thermal contact with the heating modules 1101 and 1103 and the cooling modules 1102 and 1104. Thermal insulation material may be provided between the first overshooting thermal zone and the second overshooting thermal zone, so as to avoid conduction of heat. FIG. 12 shows internal structures of the exemplary thermocycler with a cover plate 1250 placed on top of the thermal zones and below the swinging arm 1114. FIG. 13 shows an exemplary three-quarter view schematic of the exemplary thermocycler, demonstrating its internal structures from the bottom, in which a guide component 1342 may be controlled by the motor 1140. FIG. 14 shows an exemplary three-quarter view schematic of the exemplary thermocycler, demonstrating its internal structures from the bottom, wherein the guide component 1342 drives a first spindle component 1443 and a second spindle component 1444, which in turn drive the heating modules 1101 and 1103 and the cooling modules 1102 and 1104 to switch between an open position and a closed position. FIG. 15 shows an exemplary exploded three-quarter view schematic of the exemplary thermocycler.

In some embodiments, the present disclosure provides an apparatus for conducting a reaction on or using a sample. The apparatus may comprise a sample holder that holds the sample during the reaction. The reaction may comprise cycling between at least two target temperature levels including a first target temperature level and a second target temperature level. The at least two target temperature levels may include more than two target temperature levels.

The apparatus may further comprise a first overshooting thermal zone and a second overshooting thermal zone. The first overshooting thermal zone may be at a higher temperature than the first target temperature level and the second overshooting thermal zone may be at a lower temperature than the second target temperature level, or vice versa. The apparatus may also comprise a controller that is programmed to alternately and sequentially (i) place the sample holder in thermal communication with the first overshooting thermal zone to achieve the first target temperature level, and (ii) place the sample holder in thermal communication with the second overshooting thermal zone to achieve the second target temperature level.

In some embodiments, the apparatus further comprises a first translational unit and a second translational unit. The first translational unit may subject the first overshooting thermal zone and the second overshooting thermal zone to movement along a first plane, and the second translational unit may subject the sample holder to movement along a second plane that is angled with respect to the first plane. The controller may be operatively coupled to the first translational unit and the second translational unit, and the controller may be programmed to subject the first overshooting thermal zone and the second overshooting thermal zone to movement along the first plane, and subject the sample holder to movement along the second plane, to alternately and sequentially (i) place the sample holder in thermal communication with the first overshooting thermal zone to achieve the first target temperature level, and (ii) place the sample holder in thermal communication with the second overshooting thermal zone to achieve the second target temperature level. The first translational unit may subject the first overshooting thermal zone and the second overshooting thermal zone to simultaneous movement along the first plane. The movement along the second plane may be towards or away from the first plane. The second plane may be at an angle from about 45° to 90° with respect to the first plane.

In some embodiments, the controller is programmed to: (1) direct the first translational unit to move the first overshooting thermal zone to a first position and the second overshooting thermal zone to a second position along the first plane when the sample holder is raised away from the first plane; (2) direct the second translational unit to lower the sample holder towards the first plane, thereby placing the sample holder in thermal communication with the first overshooting thermal zone to achieve the first target temperature level; (3) direct the first translational unit to move the second overshooting thermal zone to the first position and the first overshooting thermal zone to a third position when the sample holder is raised away from the first plane; and (4) direct the second translational unit to lower the sample holder towards the first plane, thereby placing the sample holder in thermal communication with the second overshooting thermal zone to achieve the second target temperature level. The third position may be different than the second position. The controller may be programmed to direct the second translational unit to raise the sample holder away from the first plane between (2) and (3). The first translational unit and/or the second translational unit may include at least one motor or piezoelectric actuator. The first translational unit and/or the second translational unit may include a track. The track may be a linear track.

As an alternative, the third position may be the same as the second position. For example, the first translational unit may rotate the first overshooting thermal zone and the second overshooting thermal zone along the first plane such that the second overshooting thermal zone is at the first position and the first overshooting thermal zone is at the second position. Such rotation may be repeated to alternate the first overshooting thermal zone and the second overshooting thermal zone between the first position and the second position.

The first position, second position and third position may be discrete positions. As an alternative, the first position, second position and third position may be semi-continuous positions, which may be bounded. The first position, second position and third position may be adjustable. In some cases, additional positions may be provided, such at least 1, 2, 3, 4, 5, 6, 7, 8, 9, 10, 20, 30, 40, or 50 additional positions. Each of the additional positions may correspond to a given configuration of an overshooting thermal zone.

The first overshooting thermal zone may be at a temperature from about 110° C. to about 140° C. In some embodiments, the first overshooting thermal zone is at a temperature of at least about 120° C. or 130° C. In some embodiments, the first overshooting thermal zone is a heating unit. The second overshooting thermal zone may be at a temperature from about 0° C. to about 30° C. In some embodiments, the second overshooting thermal zone is a cooling unit. The sample holder may hold a plurality of samples.

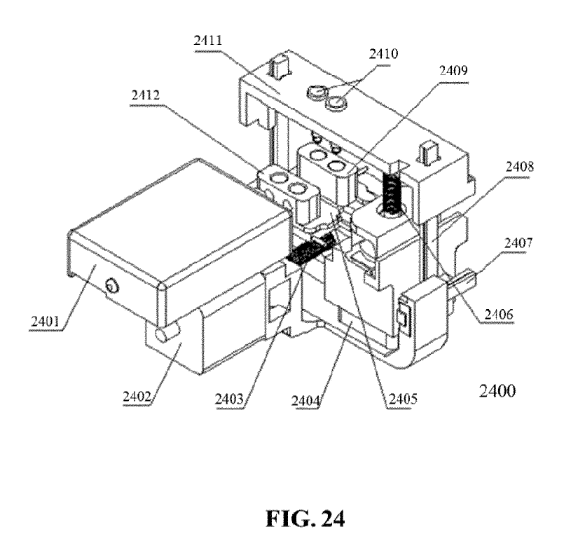

FIG. 24 shows a schematic of such an apparatus 2400. The apparatus 2400 comprises a sample holder 2411 (e.g., a tube rack) that holds one or more samples 2410 (e.g., tubes) during the reaction, a first overshooting thermal zone 2409 (e.g., a heating block) and a second overshooting thermal zone 2412 (e.g., a cooling block). The first overshooting thermal zone 2409 may be at a higher temperature than a first target temperature level and the second overshooting thermal zone 2412 may be at a lower temperature than a second target temperature level. The apparatus 2400 also comprises a first motor 2402 that drives horizontal movement of the first overshooting thermal zone 2409 and the second overshooting thermal zone 2412 along a horizontal slide guide rail 2407, aided by a horizontal lead screw 2403. The apparatus 2400 further comprises a second motor 2404 that drives vertical movement of the sample holder 2411 together with the samples 2410 along a vertical slide guide rail 2408, aided by a vertical lead screw 2406. The apparatus 2400 may also comprise a semiconductor chilling plate 2405 positioned in proximity to (e.g., below) the second overshooting thermal zone 2412, the semiconductor chilling plate 2405 may facilitate the cooling process by maintaining the second overshooting thermal zone 2412 at a low temperature (e.g., a temperature lower than the second target temperature level). The apparatus 2400 may further comprise an optical module 2401 capable of detecting signals generated from the samples 2410.

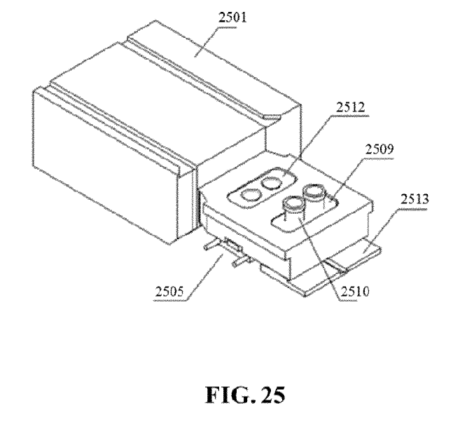

FIG. 25 provides an example of the internal structure of an apparatus. Samples 2510 may be placed in a first overshooting thermal zone 2509 (e.g. a heating block) or a second overshooting thermal zone 2512 (e.g., a cooling block). A semiconductor chilling plate 2505 may be placed below the second overshooting thermal zone 2512 to facilitate the cooling process by maintaining the second overshooting thermal zone 2512 at a low temperature (e.g., a temperature lower than the second target temperature level). A heat-insulating element (e.g., insulating cotton) may be placed below the first overshooting thermal zone 2509 and adjacent to the semiconductor chilling plate 2505, to aid in preventing or reducing heat transfer from the second overshooting thermal zone 2512 to the first overshooting thermal zone 2509.

Thermocycling Operation

Methods and devices of the present disclosure can be used in controlling a temperature of a sample precisely to achieve a desired temperature profile. A device, such as an automated thermal cycler, can be capable of controlling the temperature of a sample volume to within about plus or minus 5° C., 4° C., 3° C., 2° C., 1.2° C., 1° C., 0.7° C., 0.5° C., 0.3° C., 0.1° C., 0.05° C., 0.01° C., 0.005° C., or 0.001° C. Devices of the present disclosure can advantageously be capable of providing high quality temperature control while operating at a low voltage and/or low power.

In some embodiments, the ramping time (i.e., the time a thermal cycler takes to transition a sample volume from one temperature to another) and/or ramping rate can be important factors in amplification. For example, the temperature and time for which amplification yields a detectable amount of amplified product indicative of the presence of a target nucleic acid can vary depending upon the ramping rate and/or ramping time. The ramping rate can impact the temperature(s) and time(s) used for amplification. The ramping time and/or ramping rate may be different between cycles. In some situations, however, the ramping time and/or ramping rate between cycles can be the same. The ramping time and/or ramping rate can be adjusted based on the sample(s) that are being processed.

The ramping time and/or ramping rate of a sample can be controlled, for example, by the amount of time a sample volume is in contact with an overshooting thermal zone. An overshooting thermal zone can be used to achieve a faster rate of temperature change in a sample volume. A sample volume in thermal contact with an overshooting thermal zone can be heated or cooled to a target temperature level more rapidly than a sample volume in thermal contact with a target thermal zone. The rate at which a sample volume is heated or cooled can be controlled by how much the overshooting temperature zone differs in temperature from the target temperature level. The rate at which a sample volume is heated or cooled can be controlled by how long the sample volume is in thermal contact with an overshooting thermal zone before being moved out of contact with the overshooting thermal zone and moved into thermal contact with a target thermal zone.

In some cases, the sample volume can be brought all the way to the target temperature level through thermal contact with an overshooting thermal zone. In some cases, the sample volume can be brought part of the way to the target temperature level through thermal contact with an overshooting thermal zone, and then contacted with a target thermal zone held at the target temperature level. The sample volume can be brought 10%, 20%, 30%, 40%, 50%, 60%, 70%, 80%, 90%, 95%, 96%, 97%, 98%, 99%, 99.9%, or 100% of the way to the target temperature level through contact with an overshooting thermal zone.

In some situations, the ramping time between different temperatures can be chosen, for example, based on the nature of the sample and the reaction conditions. The temperature and incubation time can also be chosen based on the nature of the sample and the reaction conditions. In some embodiments, a single sample can be processed (e.g., subjected to amplification conditions) multiple times using multiple thermal cycles, with each thermal cycle differing for example by the ramping time, temperature, and/or incubation time. The best or optimum thermal cycle can then be chosen for that particular sample. This provides a robust and efficient method of tailoring the thermal cycles to the specific sample or combination of samples being tested.

The rate at which the sample volume is heated or cooled can be at least about 1, 2, 3, 4, 5, 6, 7, 8, 9, 10, 11, 12, 13, 14, 15, 16, 17, 18, 19, 20, 25, or 30° C./second. The rate at which the sample volume is heated or cooled can be about 1, 2, 3, 4, 5, 6, 7, 8, 9, 10, 11, 12, 13, 14, 15, 16, 17, 18, 19, 20, 25, or 30° C./second.

A target thermal zone can be used to hold a sample volume at a target temperature level for a length of time. Alternatively, a sample volume can be held at a target temperature level for a length of time by switching alternately between the state of in and out of thermal contact with the heating overshooting thermal zone. A sample volume can be held at a target temperature level for at least about 0.2, 0.5, 1, 2, 3, 4, 5, 6, 7, 8, 9, 10, 15, 20, 25, 30, 35, 40, 45, 50, 55, or 60 seconds, or for at least about 2, 3, 4, 5, 6, 7, 8, 9, 10, 15, 20, 25, 30, 35, 40, 45, 50, 55, 60, 65, 70, 75, 80, 85, or 90 minutes. A sample volume can be held at a target temperature level for at most about 0.2, 0.5, 1, 2, 3, 4, 5, 6, 7, 8, 9, 10, 15, 20, 25, 30, 35, 40, 45, 50, 55, or 60 seconds, or for at most about 2, 3, 4, 5, 6, 7, 8, 9, 10, 15, 20, 25, 30, 35, 40, 45, 50, 55, 60, 65, 70, 75, 80, 85, or 90 minutes.

Thermal cycling can be conducted for at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 cycles. Thermal cycling can be conducted for 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 cycles.

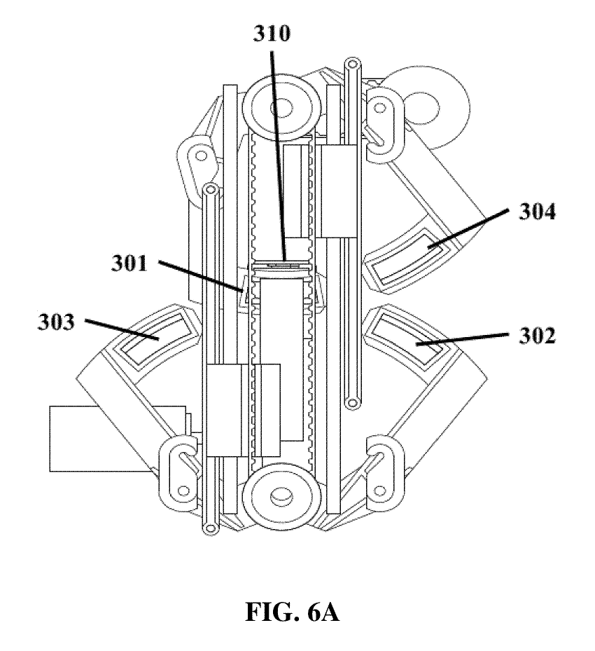

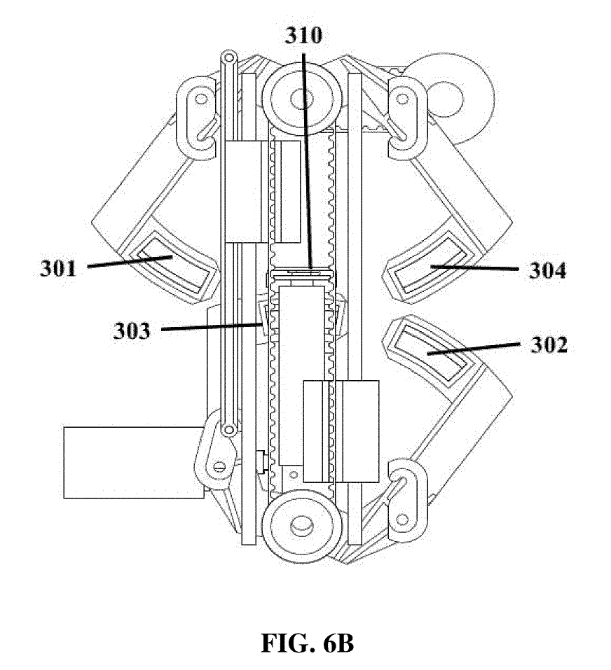

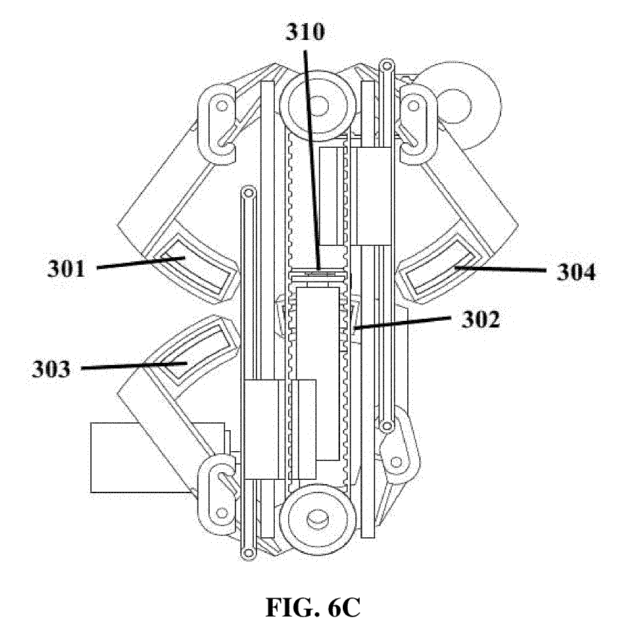

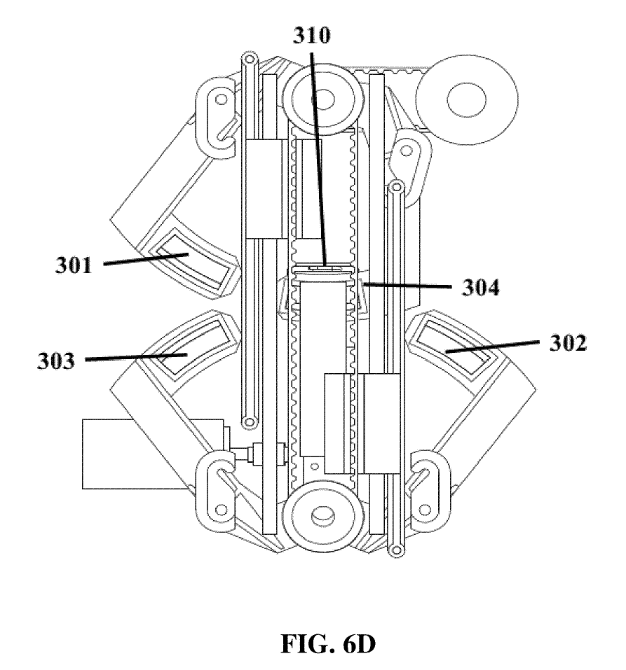

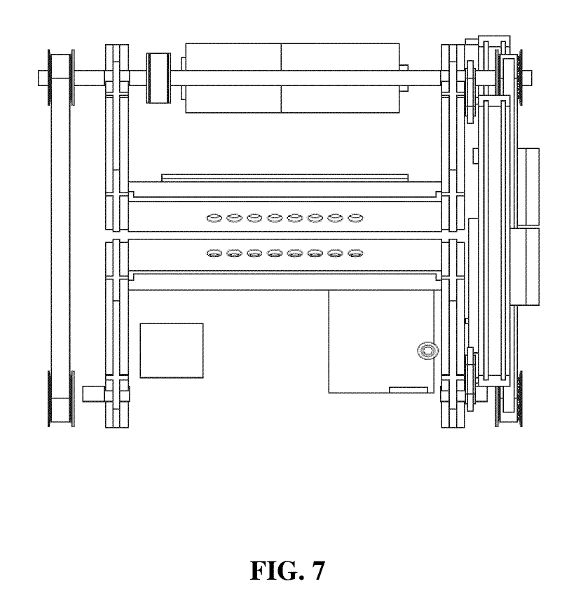

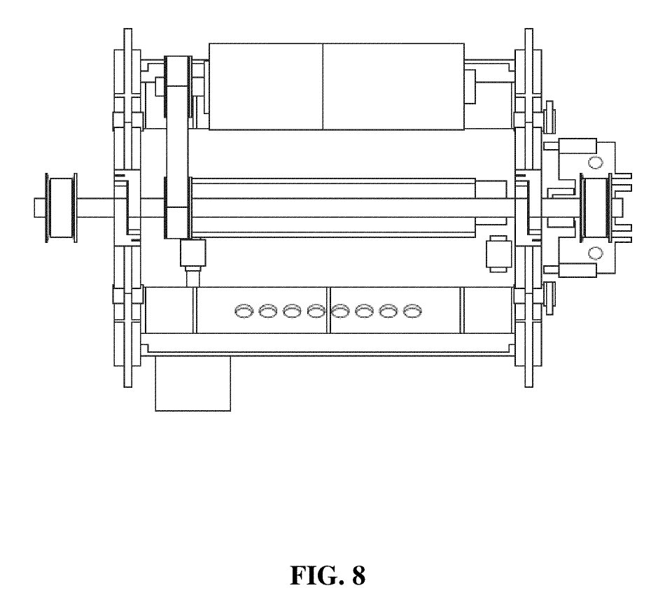

An example of thermocycler operation is shown in FIG. 6. FIG. 6A shows a sample holder 310 in thermal contact with a first overshooting thermal zone 301 on a first rotating arm. FIG. 6B shows the first overshooting thermal zone 301 moved out of thermal contact with the sample holder 310 and a first target thermal zone 303 on a second rotating arm having swung into thermal contact with the sample holder 310. FIG. 6C shows the first target thermal zone 303 moved out of thermal contact with the sample holder 310 and a second overshooting thermal zone 302 on a third rotating arm having swung into thermal contact with the sample holder 310. FIG. 6D shows the second overshooting thermal zone 302 moved out of thermal contact with the sample holder 310 and a second target thermal zone 304 on a fourth rotating arm having swung into thermal contact with the sample holder 310. The thermocycler can then return to the configuration shown in FIG. 6A and can repeat the cycle as many times as needed. The sample holder can be capable of moving in coordination with the rotating arms. For example, the sample holder can move up vertically as a thermal zone on a rotating arm moves away horizontally, and then the sample holder can move down to meet the next thermal zone as the next thermal zone moves in horizontally on its rotating arm. Additional views of a thermocycler are shown in FIG. 7 and FIG. 8.

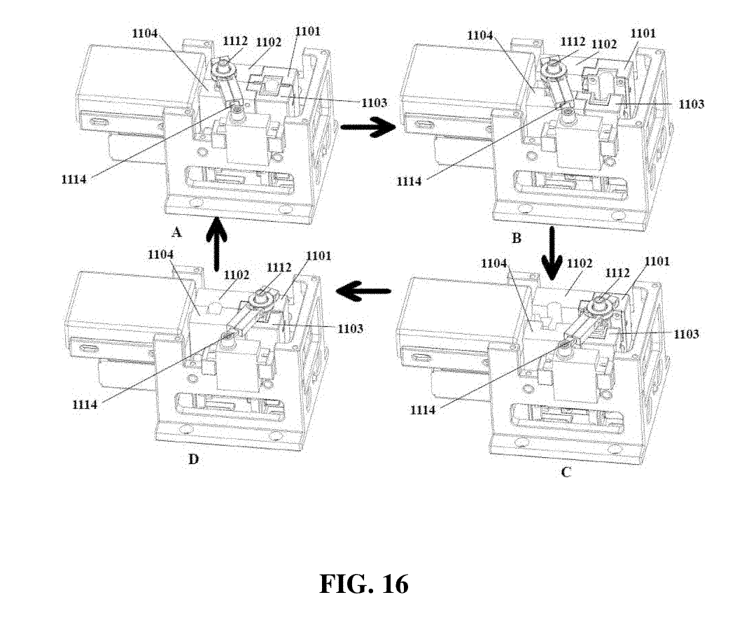

Another example of thermocycler operation is shown in FIG. 16. FIG.16 (panel A) shows a reaction vessel 1112 controlled by a swinging arm 1114 placed in thermal contact with a cooling overshooting thermal zone composed of a first cooling module 1102 and a second cooling module 1104. FIG. 16 (panel B) shows the first cooling module 1102 and the second cooling module 1104 moved to an open position and out of thermal contact the reaction vessel 1112. FIG. 16 (panel C) shows the reaction vessel 1112 driven by the swinging arm 1114 having swung into a heating overshooting thermal zone comprised by a first heating module 1101 and a second heating module 1103. The first heating module 1101 and the second heating module 1103 may be in an open position. FIG. 16 (panel D) shows the first heating module 1101 and the second heating module 1103 moved to a closed position surrounding and in thermal contact with the reaction vessel. The thermocycler can then return to the configuration shown in FIG. 16 (panel A) and can repeat the cycle as many times as needed.

The timing of the motion of moveable elements coupled to thermal zones or sample holders can be controlled by a timing control system. For example, the thermal zones and/or the sample holder can be mounted on moveable elements, and these moveable elements can be connected to one or more motors. The moveable elements can be driven by a single motor, belt, or other driver. The moveable elements can each have separate motors or other drivers. The timing control system can be electronic or mechanical.

The movement of the moveable elements can be controlled by an electronic timing control system. An electronic timing control system can comprise one or more computer processors. The electronic timing control system can be operated to move the thermal zones into and out of thermal contact with sample volumes in a determined order and for determined amounts of time.

The timing control system can be mechanical. For example, the thermal zones and/or the sample holder can be mounted on moveable elements, and these moveable elements can be connected to a mechanical timing control system such as a belt or cam. The moveable elements can be connected to the mechanical timing control system such that, when the mechanical timing control system is operated, the moveable elements move the thermal zones into and out of thermal contact with sample volumes in a determined order and for determined amounts of time.

The amount of time each thermal zone is placed in thermal contact with a sample volume per cycle can be the same or different for the thermal zones present in a thermocycler. A thermal zone can be in thermal contact with a sample volume for about 0.1, 0.2, 0.3, 0.4, 0.5, 0.6, 0.7, 0.7, 0.8, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 35, 40, 45, 50, or 55 seconds, or for about 1, 1.5, 2, 2.5, 3, 3.5, 4, 4.5, or 5 minutes or more.

The time to complete a single thermal cycle can be less than or equal to about 10 minutes, 5 minutes, 1 minute, 50 seconds, 40 seconds, 30 seconds, 20 seconds, 15 seconds, 10 seconds, 9 seconds, 8 seconds, 7 seconds, 6 seconds, 5 seconds, 4 seconds 3 seconds, 2 seconds, 1 second, 0.9 seconds, 0.8 seconds, 9.7 seconds, 0.6 seconds, 0.5 seconds, 0.4 seconds, 0.3 seconds, 0.2 seconds, or 0.1 seconds.

The timing control system can be programmable or adjustable. Aspects of the thermocycler cycle can be adjusted or programmed, such as the number of temperature levels, the temperature of a given temperature level, the order in which the sample volume is brought to the temperature levels, the time spent to move from one temperature level to another, the amount of time a sample volume spends at a given temperature level, the number of cycles conducted, or other parameters.

In another aspect, for thermocycler operation, another apparatus of the present disclosure may be employed. The apparatus may comprise a sample holder that holds the sample during a reaction. The reaction comprises cycling between at least two target temperature levels including a first target temperature level and a second target temperature level. The at least two target temperature levels may include more than two target temperature levels. The apparatus may further comprise a first overshooting thermal zone and a second overshooting thermal zone. The first overshooting thermal zone may be at a higher temperature than the first target temperature level and the second overshooting thermal zone may be at a lower temperature than the second target temperature level, or vice versa. The apparatus may also comprise a controller that is programmed to alternately and sequentially (i) place the sample holder in thermal communication with the first overshooting thermal zone to achieve the first target temperature level, and (ii) place the sample holder in thermal communication with the second overshooting thermal zone to achieve the second target temperature level.

In some embodiments, the apparatus further comprises a first translational unit and a second translational unit. The first translational unit may subject the first overshooting thermal zone and the second overshooting thermal zone to movement along a first plane, and the second translational unit may subject the sample holder to movement along a second plane that is angled with respect to the first plane. The controller may be operatively coupled to the first translational unit and the second translational unit, and the controller may be programmed to subject the first overshooting thermal zone and the second overshooting thermal zone to movement along the first plane, and subject the sample holder to movement along the second plane, to alternately and sequentially (i) place the sample holder in thermal communication with the first overshooting thermal zone to achieve the first target temperature level, and (ii) place the sample holder in thermal communication with the second overshooting thermal zone to achieve the second target temperature level. The first translational unit may subject the first overshooting thermal zone and the second overshooting thermal zone to simultaneous movement along the first plane. The movement along the second plane may be towards or away from the first plane. The second plane may be at an angle from about 45° to 90° with respect to the first plane.

In some embodiments, the controller is programmed to: (1) direct the first translational unit to move the first overshooting thermal zone to a first position and the second overshooting thermal zone to a second position along the first plane when the sample holder is raised away from the first plane; (2) direct the second translational unit to lower the sample holder towards the first plane, thereby placing the sample holder in thermal communication with the first overshooting thermal zone to achieve the first target temperature level; (3) direct the first translational unit to move the second overshooting thermal zone to the first position and the first overshooting thermal zone to a third position when the sample holder is raised away from the first plane; and (4) direct the second translational unit to lower the sample holder towards the first plane, thereby placing the sample holder in thermal communication with the second overshooting thermal zone to achieve the second target temperature level. The third position may be different than the second position. The controller may be programmed to direct the second translational unit to raise the sample holder away from the first plane between (2) and (3). The first translational unit and/or the second translational unit may include at least one motor or piezoelectric actuator. The first translational unit and/or the second translational unit may include a track. The track may be a linear track.

The first position, second position and third position may be discrete positions. As an alternative, the first position, second position and third position may be semi-continuous positions, which may be bounded. The first position, second position and third position may be adjustable. In some cases, additional positions may be provided, such at least 1, 2, 3, 4, 5, 6, 7, 8, 9, 10, 20, 30, 40, or 50 additional positions. Each of the additional positions may correspond to a given configuration of an overshooting thermal zone.

The first overshooting thermal zone may be at a temperature from about 110° C. to about 140° C. In some embodiments, the first overshooting thermal zone is at a temperature of at least about 120° C. or 130° C. In some embodiments, the first overshooting thermal zone is a heating unit. The second overshooting thermal zone may be at a temperature from about 0° C. to about 30° C. In some embodiments, the second overshooting thermal zone is a cooling unit. The sample holder may hold a plurality of samples.

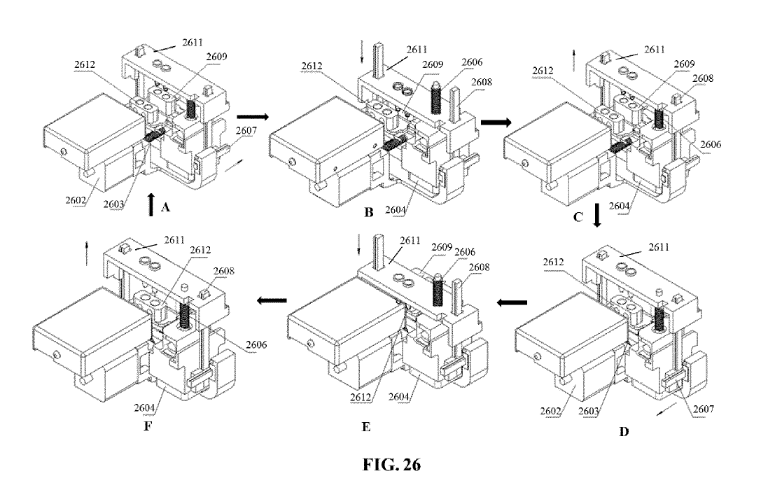

An example is demonstrated in FIG. 26. The apparatus comprises a sample holder 2611 that holds one or more vials (or tubes), each vial having one or more samples during the reaction. In the illustrated example, the sample holder 2611 holds two vials comprising samples. The reaction comprises cycling between at least two target temperature levels including a first target temperature level and a second target temperature level. The first target temperature level may be lower than a temperature of the first overshooting thermal zone 2609. The second target temperature level may be higher than a temperature of the second overshooting thermal zone 2612.

The apparatus further comprises a first overshooting thermal zone 2609 (e.g., a heating block) and a second overshooting thermal zone 2612 (e.g., a cooling block), wherein the first overshooting thermal zone is at a higher temperature than the first target temperature level and the second overshooting thermal zone is at a lower temperature than the second target temperature level. The apparatus further comprises a first translational unit and a second translational unit. The first translational unit comprises a first motor 2602, a horizontal first lead screw 2603 and a horizontal first slide guide rail 2607. The second translational unit comprises a second motor 2604, a vertical second lead screw 2606 and a vertical second slide guide track 2608. In FIG. 26 (panel A), the sample holder 2611 is above the first overshooting thermal zone 2609 in a first position. The second overshooting thermal zone 2612 is in a second position. With the operation of the second motor 2604, the sample holder 2611 is lowered towards the first overshooting thermal zone 2609 along the vertical second slide guide track 2608 and aided by the vertical second lead screw 2606, thereby placing the samples in thermal communication (e.g., thermal contact) with the first overshooting thermal zone 2609 for a time period sufficient to achieve a first target temperature level, as shown in FIG. 26 (panel B). After achieving the first target temperature level, the sample holder is raised away from the first overshooting thermal zone 2609 along the vertical second slide guide track 2608 and aided by the vertical second lead screw 2606, as shown in FIG. 26 (panel C). Then, the first motor 2602 moves the first overshooting thermal zone 2609 to a third position and the second overshooting thermal zone 2612 to the first position along the horizontal first slide guide rail 2607 and aided by the horizontal first lead screw 2603, placing the sample holder 2611 above the second overshooting thermal zone 2612, as shown in FIG. 26 (panel D). Next, the sample holder 2611 is lowered towards the second overshooting thermal zone 2612 along the vertical second slide guide track 2608 and aided by the vertical second lead screw 2606, thereby placing the samples in thermal communication with the second overshooting thermal zone 2612 for a time period sufficient to achieve a second target temperature level, as shown in FIG. 26 (panel E). After achieving the second target temperature level, the sample holder is raised away from the second overshooting thermal zone 2612 along the vertical second slide guide track 2608 and aided by the vertical second lead screw 2606, as shown in FIG. 26 (panel F). Then, the first motor 2602 moves the second overshooting thermal zone 2609 back to the first position and the first overshooting thermal zone 2612 back to the third position along the horizontal first slide guide rail 2607 and aided by the horizontal first lead screw 2603, placing the sample holder 2611 above the first overshooting thermal zone 2612, as shown in FIG. 26 (panel A). This process may be repeated as many times as necessary.

As an alternative, the third position may be the same as the second position. For example, with respect to FIG. 26 (panel D), the first motor 2602 may rotate the first overshooting thermal zone and the second overshooting thermal zone along a plane such that the second overshooting thermal zone moves to the first position and the first overshooting thermal zone moves to the second position. Such rotation may be repeated to alternate the first overshooting thermal zone and the second overshooting thermal zone between the first position and the second position.

The first position, second position and third position may be discrete positions. As an alternative, the first position, second position and third position may be semi-continuous positions, which may be bounded. The first position, second position and third position may be adjustable. In some cases, additional positions may be provided, such at least 1, 2, 3, 4, 5, 6, 7, 8, 9, 10, 20, 30, 40, or 50 additional positions. Each of the additional positions may correspond to a given configuration of an overshooting thermal zone.

The temperature of a sample in a vial held by the sample holder 2611 may be monitored to achieve the target temperature levels. For example, the temperature of the sample may be monitored by measuring infrared radiation (IR) from the vial or using a thermocouple. As an alternative, the temperature may not be monitored, but the time period the vial is in thermal communication with the first overshooting thermal zone 2609 and the second overshooting thermal zone 2612 may be selected to achieve a programmed sample temperature profile with time.

For example, a target nucleic acid molecule in a sample (the sample may comprise e.g., purified DNA and/or RNA, pseudoviruses, serum, plasma, whole blood, stool sample, or swab sample, etc.) may be amplified and detected with the method, system and/or apparatus of the present disclosure, using a predetermined amplification protocol. The protocol may comprise: 1) heating the sample at a first overshooting temperature of about 115° C., until a first target temperature of about 94° C. is reached; 2) then, cooling the sample at a second overshooting temperature of about 20° C., until a second target temperature of about 48° C. is reached; and 3) repeating operations 1) and 2) as necessary.

Samples

Reactions and other processes conducted with the present methods, devices, apparatuses, and systems can be conducted on one or more samples. A sample can comprise a target nucleic acid. A sample can comprise an agent that detects amplified target nucleic acid (e.g., a detectable nucleic acid binding agent). A sample can comprise reagents for conducting nucleic acid amplification. Depending on the nature of the target nucleic acid that is to be amplified, reagents can comprise reverse transcriptase for conducting reverse-transcriptase coupled PCT, dNTPs, or Mg²⁺ ions.

The sample can be a biological sample. The biological sample can be taken from a subject. For example, the sample can be taken from a living subject directly. In some embodiments, the biological sample can include breath, blood, urine, feces, saliva, cerebrospinal fluid and sweat. Any suitable biological sample that comprises nucleic acid can be obtained from a subject. A biological sample can be solid matter (e.g., biological tissue) or can be a fluid (e.g., a biological fluid). In general, a biological fluid can include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues.

A subject can be a living subject or a dead subject. The subject can be a human or an animal. In some examples, the subject can be mammal. Examples of subjects can include, but are not limited to simians, avines, canines, felines, equines, bovines, ovines, porcines, delphines, rodents (e.g., mice, rats), or insects.

A biological sample can be obtained from a subject by various approaches. Non-limiting examples of such approaches to obtain a biological sample directly from a subject include accessing the circulatory system (e.g., intravenously or intra-arterially via a syringe or other needle), collecting a secreted biological sample (e.g., feces, urine, sputum, saliva, etc.), surgically (e.g., biopsy), swabbing (e.g., buccal swab, oropharyngeal swab), pipetting, and breathing. Moreover, a biological sample can be obtained from any anatomical part of a subject where a desired biological sample is located.

A biological sample obtained directly from a subject can generally refer to a biological sample that has not been further processed after being obtained from the subject, with the exception of any approach used to collect the biological sample from the subject for further processing. For example, blood is obtained directly from a subject by accessing the subject's circulatory system, removing the blood from the subject (e.g., via a needle), and entering the removed blood into a receptacle. The receptacle can comprise reagents (e.g., anti-coagulants) such that the blood sample is useful for further analysis. In another example, a swab can be used to access epithelial cells on an oropharyngeal surface of the subject. After obtaining the biological sample from the subject, the swab containing the biological sample can be contacted with a fluid (e.g., a buffer) to collect the biological fluid from the swab. Alternatively, pre-processing can occur on the biological sample prior to being provided to the device.

In some embodiments, a biological sample has not been purified when provided in a reaction vessel. In some embodiments, the nucleic acid of a biological sample has not been extracted when the biological sample is provided to a reaction vessel. For example, the RNA or DNA in a biological sample may not be extracted from the biological sample when providing the biological sample to a reaction vessel. Moreover, in some embodiments, a target nucleic acid (e.g., a target RNA or target DNA) present in a biological sample may not be concentrated prior to providing the biological sample to a reaction vessel. Alternatively, dilution or concentration of the sample can occur prior to being provided to a device.

The sample can have a target nucleic acid to be amplified. The target nucleic acid can be amplified to generate an amplified product. A target nucleic acid can be a target RNA or a target DNA. In cases where the target nucleic acid is a target RNA, the target RNA can be any type of RNA. In some embodiments, the target RNA is viral RNA. In some embodiments, the viral RNA can be pathogenic to the subject. Non-limiting examples of pathogenic viral RNA include human immunodeficiency virus I (HIV I), human immunodeficiency virus II (HIV II), orthomyxoviruses, influenza viruses (e.g., H1N1 , H3N2, H5N1, H7N9), hepevirus, hepatitis A, hepatitis B, hepatitis C, hepatitis D, hepatitis E, hepatitis G, Epstein-Barr virus, mononucleosis, cytomegalovirus, SARS, West Nile Fever, Ebola virus, polio, and measles.

In cases where the target nucleic acid is a target DNA, the target DNA can be any type of DNA. In some embodiments, the target DNA is viral DNA. In some embodiments, the viral DNA can be pathogenic to the subject. Non-limiting examples of DNA viruses include herpes simplex virus, smallpox, and chickenpox. In some cases, a target DNA can be a parasite DNA, such as malaria parasite or plasmodium. In some cases, a target DNA can be a bacterial DNA. The bacterial DNA can be from a bacterium pathogenic to the subject such as, for example, Mycobacterium tuberculosis—a bacterium known to cause tuberculosis.

The sample can also include an agent that detects amplified target nucleic acid. The agent can be a reporter agent that can yield a detectable signal whose presence or absence is indicative of the presence of an amplified product. The intensity of the detectable signal can be proportional to the amount of amplified product. For example, the detectable signal can be directly linearly proportional, exponentially proportional, reversely proportional, or have any other type of proportional relationship to the amount of amplified product. In some cases, where amplified product is generated of a different type of nucleic acid than the target nucleic acid initially amplified, the intensity of the detectable signal can be proportional to the amount of target nucleic acid initially amplified. For example, in the case of amplifying a target RNA via parallel reverse transcription and amplification of the DNA obtained from reverse transcription, reagents necessary for both reactions can also comprise a reporter agent can yield a detectable signal that is indicative of the presence of the amplified DNA product and/or the target RNA amplified. The intensity of the detectable signal can be proportional to the amount of the amplified DNA product and/or the original target RNA amplified. The use of a reporter agent also enables real-time amplification methods, including real-time PCR for DNA amplification.

Reporter agents can be linked with nucleic acids, including amplified products, by covalent or non-covalent linkages. Non-limiting examples of non-covalent linkages include ionic interactions, Van der Waals forces, hydrophobic interactions, hydrogen bonding, and combinations thereof. In some embodiments, reporter agents can bind to initial reactants and changes in reporter agent levels can be used to detect amplified product. In some embodiments, reporter agents can only be detectable (or non-detectable) as nucleic acid amplification progresses. In some embodiments, an optically-active dye (e.g., a fluorescent dye) can be used as can be used as a reporter agent. An agent for detecting amplified target nucleic acid can be a nucleic acid binding dye. The dye can be a DNA-intercalating dye. Non-limiting examples of dyes include Eva green, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and-2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, Eva Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5-(or 6-) iodoacetamidofluorescein, 5-{[2(and 3)-5-(Acetylmercapto)-succinyl] amino } fluorescein (SAMS A-fluorescein), lis samine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some instances, a reporter agent can be a sequence-specific oligonucleotide probe that can be optically active when hybridized with an amplified product. Due to sequence-specific binding of the probe to the amplified product, use of oligonucleotide probes can increase specificity and sensitivity of detection. A probe can be linked to any of the optically-active reporter agents (e.g., dyes) described herein and can also include a quencher capable of blocking the optical activity of an associated dye. Non-limiting examples of probes that can be useful used as reporter agents include TaqMan probes, TaqMan Tamara probes, TaqMan MGB probes, or Lion probes.

A reporter agent can be an RNA oliognucleotide probe that can include an optically-active dye (e.g., fluorescent dye) and a quencher positioned adjacently on the probe. The close proximity of the dye with the quencher can block the optical activity of the dye. The probe can bind to a target sequence to be amplified. Upon the breakdown of the probe with the exonuclease activity of a DNA polymerase during amplification, the quencher and dye are separated, and the free dye regains its optical activity that can subsequently be detected.

A reporter agent may be a molecular beacon. A molecular beacon can include, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and can be detected. The sequence specificity of the molecular beacon for a target sequence on the amplified product can improve specificity and sensitivity of detection.

In some embodiments, a reporter agent can be a radioactive species. Non-limiting examples of radioactive species include ¹⁴C, ¹²⁸I, ¹²⁴I, ¹²⁵I, ¹³¹I, Tc99m, ³⁵S, or ³H.

In some embodiments, a reporter agent can be an enzyme that is capable of generating a detectable signal. Detectable signal can be produced by activity of the enzyme with its substrate or a particular substrate in the case the enzyme has multiple substrates. Non-limiting examples of enzymes that can be used as reporter agents include alkaline phosphatase, horseradish peroxidase, I²-galactosidase, alkaline phosphatase, β-galactosidase, acetylcholinesterase, and luciferase.

The sample can be provided with reagents necessary for nucleic acid amplification within the device. In some instances, a reagent can comprise one or more of the following: (i) a reverse transcriptase, (ii) a DNA polymerase, and (iii) a primer set for the target nucleic acid (e.g., RNA). Some examples of reagents can include a commercially available pre-mixture (e.g., Qiagen One-Step RT-PCR or One-Step RT-qPCR kit) comprising reverse transcriptases (e.g., Sensiscript and Omniscript transcriptases), a DNA Polymerase (e.g., HotStarTaq DNA Polymerase), and dNTPs.

In some instances, the sample can be provided within a sample container, such as a reaction vessel. Any components of the sample including the target nucleic acid, agent that detects amplified target nucleic acid, and/or reagents for nucleic acid amplification can be provided within the reaction vessel to obtain a reaction mixture. Any suitable reaction vessel can be used. In some embodiments, a reaction vessel comprises a body that can include an interior surface, an exterior surface, an open end, and an opposing closed end. In some embodiments, a reaction vessel can comprise a cap. The cap can be configured to contact the body at its open end, such that when contact is made the open end of the reaction vessel is closed. In some cases, the cap is permanently associated with the reaction vessel such that it remains attached to the reaction vessel in open and closed configurations. In some cases, the cap is removable, such that when the reaction vessel is open, the cap is separated from the reaction vessel. In some embodiments, a reaction vessel can be sealed, in some cases hermetically sealed. The reaction vessel can be fluid-tight.

A reaction vessel can be of varied size, shape, weight, and configuration. In some examples, a reaction vessel can be round or oval tubular shaped. In some embodiments, a reaction vessel can be rectangular, square, diamond, circular, elliptical, or triangular shaped. A reaction vessel can be regularly shaped or irregularly shaped. In some embodiments, the closed end of a reaction vessel can have a tapered, rounded, or flat surface. For example, a flat cap, rounded, cap, or tapered cap can be provided. Non-limiting examples of types of a reaction vessel include a tube, a well, a capillary tube, a cartridge, a cuvette, a centrifuge tube, or a pipette tip.

Any dimensions can be provided for a reaction vessel. The reaction vessel can be configured to contain at least about 0.2 microliters (mL) or 0.5 mL of sample. The reaction vessel can be configured to contain at least about 1 mL, 1.5 mL, 2 mL, 2.5 mL, 3 mL, 3.5 mL, 4 mL, 5 mL, 6 mL, 7 mL, 8 mL, 9 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 90 mL, 95 mL, 100 mL, 110 mL, 120 mL, 120 mL, 140 mL, 150 mL, 160 mL, 170 mL, 180 mL, 190 mL, 200 mL, 250 mL, 300 mL, 350 mL, 400 mL, 450 mL, or 500 mL. The reaction vessel can be configured to contain at most about 1 mL, 1.5 mL, 2 mL, 2.5 mL, 3 mL, 3.5 mL, 4 mL, 5 mL, 6 mL, 7 mL, 8 mL, 9 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 90 mL, 95 mL, 100 mL, 110 mL, 120 mL, 120 mL, 140 mL, 150 mL, 160 mL, 170 mL, 180 mL, 190 mL, 200 mL, 250 mL, 300 mL, 350 mL, 400 mL, 450 mL, or 500 mL. The reaction vessel can be configured to contain about 1 mL, 1.5 mL, 2 mL, 2.5 mL, 3 mL, 3.5 mL, 4 mL, 5 mL, 6 mL, 7 mL, 8 mL, 9 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 90 mL, 95 mL, 100 mL, 110 mL, 120 mL, 120 mL, 140 mL, 150 mL, 160 mL, 170 mL, 180 mL, 190 mL, 200 mL, 250 mL, 300 mL, 350 mL, 400 mL, 450 mL, or 500 mL. The reaction vessel can have a volume configured to contain no more than a volume falling into a range between two of the values described herein. The reaction vessel can have a volume from about 20 mL to about 200 mL. The reaction vessel can have a volume from about 50 mL to about 200 mL. The reaction vessel can have a volume from about 100 mL to about 200 mL.

The reaction vessel can have a height of at least about 0.25 centimeters (cm), 0.5 cm, 0.75 cm, 1 cm, 1.25 cm, 1.5 cm, 1.75 cm, 2 cm, 2.25 cm, 2.5 cm, 2.75 cm, 3 cm, 3.25 cm, 3.5 cm, 3.75 cm, 4 cm, 4.25 cm, 4.5 cm, 4.75 cm, 5 cm, 6 cm, 7 cm, 8 cm, 9 cm, or 10 cm. The reaction vessel can have a height of at most about 0.25 centimeters (cm), 0.5 cm, 0.75 cm, 1 cm, 1.25 cm, 1.5 cm, 1.75 cm, 2 cm, 2.25 cm, 2.5 cm, 2.75 cm, 3 cm, 3.25 cm, 3.5 cm, 3.75 cm, 4 cm, 4.25 cm, 4.5 cm, 4.75 cm, 5 cm, 6 cm, 7 cm, 8 cm, 9 cm, or 10 cm. The reaction vessel can have a height of about 0.25 centimeters (cm), 0.5 cm, 0.75 cm, 1 cm, 1.25 cm, 1.5 cm, 1.75 cm, 2 cm, 2.25 cm, 2.5 cm, 2.75 cm, 3 cm, 3.25 cm, 3.5 cm, 3.75 cm, 4 cm, 4.25 cm, 4.5 cm, 4.75 cm, 5 cm, 6 cm, 7 cm, 8 cm, 9 cm, or 10 cm. The reaction vessel can have a height greater than any of the values described herein. The reaction vessel can have a height falling into a range between any two of the values described herein.

The reaction vessel can have a cross-sectional area of at least about 0.25 square centimeters (cm²), 0.5 cm², 0.75 cm², 1 cm², 1.25 cm², 1.5 cm², 1.75 cm², 2 cm², 2.25 cm², 2.5 cm², 2.75 cm², 3 cm², 3.25 cm², 3.5 cm², 3.75 cm², 4 cm², 4.25 cm², 4.5 cm², 4.75 cm², or 5 cm². The reaction vessel can have a cross-sectional area of at most about 0.25 square centimeters (cm²), 0.5 cm², 0.75 cm², 1 cm², 1.25 cm², 1.5 cm², 1.75 cm², 2 cm², 2.25 cm², 2.5 cm², 2.75 cm², 3 cm², 3.25 cm², 3.5 cm², 3.75 cm², 4 cm², 4.25 cm², 4.5 cm², 4.75 cm², or 5 cm². The reaction vessel can have a cross-sectional area of about 0.25 square centimeters (cm²), 0.5 cm², 0.75 cm², 1 cm², 1.25 cm², 1.5 cm², 1.75 cm², 2 cm², 2.25 cm², 2.5 cm², 2.75 cm², 3 cm², 3.25 cm², 3.5 cm², 3.75 cm², 4 cm², 4.25 cm², 4.5 cm², 4.75 cm², or 5 cm². The reaction vessel can have a cross-sectional area less than any of the values described herein. The reaction vessel can have a cross-sectional area falling into a range between any two of the values described herein.

Reaction vessels can be constructed of any suitable material with non-limiting examples of such materials that include glasses, metals, plastics, and combinations thereof. Reaction vessels can be made from optically transparent or translucent materials that can permit an optical signal from within the reaction vessel to leave the reaction vessel. The reaction vessels can be made from a material that may or may not filter an optical signal exiting the reaction vessel. In some instances, the reaction vessels can be formed from a clear material that can permit a detector to view the interior of the reaction vessels. In some instances, the interior of the reaction vessels can be imaged. Alternatively, an amount of optical signal exiting the reaction vessel can be detected and measured.

A thermal cycler can be capable of receiving a reaction vessel. The reaction vessels can be removably provided to the thermal cycler. The reaction vessels can be inserted within a device or taken out of the device. The reaction vessels can be placed onto a supporting component of the thermal cycler or taken off the supporting component. In alternative embodiments, the sample can be loaded directly into the device without requiring a separate reaction vessel. In some instances, reaction vessels or receptacles can be directly built-into the device. A sample holder can hold at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 sample volumes.

Sample volumes (e.g., reaction vessels) or sample holders can be brought into thermal contact with thermal zones through a variety of motions. Thermal zones can be moved into contact with sample volumes, or sample volumes can be moved into contact with thermal zones. In some cases, sample volumes can be mounted on or otherwise coupled to moveable elements, such as arms, belts, cams, discs, levers, tracks, or wheels. Such moveable elements can be driven by one or more motors, springs, or other driving elements.

A device of the present disclosure (e.g., a thermocycler) can accept the reaction vessel having the sample therein, or can directly receive the sample. The thermal cycler can be capable of alternatingly heating and cooling the sample. Multiple cycles of heating and cooling can be provided. Any temperature profile can be provided for the various heating and cooling cycles.

Nucleic Acid Amplification

Devices and methods of the present disclosure can be used to conduct processes and reactions requiring cycling between at least two temperature levels, such as nucleic acid amplification. Reactions described herein can be conducted in parallel, in some implementations. Parallel amplification reactions can be amplification reactions that can occur in the same reaction vessel and at the same time. Parallel nucleic acid amplification reactions can be conducted, for example, by including reagents necessary for each nucleic acid amplification reaction in a reaction vessel to obtain a reaction mixture and subjecting the reaction mixture to conditions necessary for each nucleic amplification reaction. For example, reverse transcription amplification and DNA amplification can be conducted in parallel, by providing reagents necessary for both amplification methods in a reaction vessel to form to obtain a reaction mixture and subjecting the reaction mixture to conditions suitable for conducting both amplification reactions. DNA generated from reverse transcription of the RNA can be amplified in parallel to generate an amplified DNA product. Any suitable number of nucleic acid amplification reactions can be conducted in parallel. In some cases, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 nucleic acid amplification reactions are conducted in parallel.

Any type of nucleic acid amplification reaction can be used to amplify a target nucleic acid and generate an amplified product. Moreover, amplification of a nucleic acid can be linear, exponential, or a combination thereof. Amplification can be emulsion based or can be non-emulsion based. Non-limiting examples of nucleic acid amplification methods include reverse transcription, primer extension, polymerase chain reaction, ligase chain reaction, helicase-dependent amplification, asymmetric amplification, rolling circle amplification, and multiple displacement amplification (MDA). In some embodiments, the amplified product can be DNA. In cases where a target RNA is amplified, DNA can be obtained by reverse transcription of the RNA and subsequent amplification of the DNA can be used to generate an amplified DNA product. The amplified DNA product can be indicative of the presence of the target RNA in the biological sample. In cases where DNA is amplified, any DNA amplification method known in the art can be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some cases, DNA amplification is linear. In some cases, DNA amplification is exponential. In some cases, DNA amplification is achieved with nested PCR, which can improve sensitivity of detecting amplified DNA products.

In any of the various aspects, primer sets directed to a target nucleic acid can be utilized to conduct nucleic acid amplification reaction. Primer sets generally comprise one or more primers. For example, a primer set can comprise about 1, 2, 3, 4, 5, 6, 7, 8, 9, 10, or more primers. In some cases, a primer set or can comprise primers directed to different amplified products or different nucleic acid amplification reactions. For example, a primer set can comprise a first primer necessary to generate a first strand of nucleic acid product that is complementary to at least a portion of the target nucleic acid and a second primer complementary to the nucleic acid strand product necessary to generate a second strand of nucleic acid product that is complementary to at least a portion of the first strand of nucleic acid product.

For example, a primer set can be directed to a target RNA. The primer set can comprise a first primer that can be used to generate a first strand of nucleic acid product that is complementary to at least a portion the target RNA. In the case of a reverse transcription reaction, the first strand of nucleic acid product can be DNA. The primer set can also comprise a second primer that can be used to generate a second strand of nucleic acid product that is complementary to at least a portion of the first strand of nucleic acid product. In the case of a reverse transcription reaction conducted in parallel with DNA amplification, the second strand of nucleic acid product can be a strand of nucleic acid (e.g., DNA) product that is complementary to a strand of DNA generated from an RNA template.

Where desired, any suitable number of primer sets can be used. For example, about 1, 2, 3, 4, 5, 6, 7, 8, 9, 10, or more primer sets can be used. Where multiple primer sets are used, one or more primer sets can each correspond to a particular nucleic acid amplification reaction or amplified product.

In some embodiments, a DNA polymerase is used. Any suitable DNA polymerase can be used, including commercially available DNA polymerases. A DNA polymerase generally refers to an enzyme that is capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes can be required, which can change the thermal profile based on different polymerases.

A reverse transcriptase is used can be used in accordance with some embodiments of the invention. Any suitable reverse transcriptase can be used. A reverse transcriptase generally refers to an enzyme that is capable of incorporating nucleotides to a strand of DNA, when bound to an RNA template. Non-limiting examples of reverse transcriptases include HIV-1 reverse transcriptase, M-MLV reverse transcriptase, AMV reverse transcriptase, telomerase reverse transcriptase, and variants, modified products and derivatives thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration.

Denaturation temperatures can vary depending upon, for example, the particular biological sample analyzed, the particular source of target nucleic acid (e.g., viral particle, bacteria) in the biological sample, the reagents used, and/or the desired reaction conditions. For example, a denaturation temperature can be from about 80° C. to about 110° C. In some examples, a denaturation temperature can be from about 90° C. to about 100° C. In some examples, a denaturation temperature can be from about 87° C. to about 95° C. In some examples, a denaturation temperature can be from about 90° C. to about 97° C. In some examples, a denaturation temperature can be from about 92° C. to about 95° C. In still other examples, a denaturation temperature can be at least or about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Denaturation durations can vary depending upon, for example, the particular biological sample analyzed, the particular source of target nucleic acid (e.g., viral particle, bacteria) in the biological sample, the reagents used, and/or the desired reaction conditions. For example, a denaturation duration can be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, a denaturation duration can be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Elongation temperatures can vary depending upon, for example, the particular biological sample analyzed, the particular source of target nucleic acid (e.g., viral particle, bacteria) in the biological sample, the reagents used, and/or the desired reaction conditions. For example, an elongation temperature can be from about 30° C. to about 80° C. In some examples, an elongation temperature can be from about 35° C. to about 72° C. In some examples, an elongation temperature can be from about 40° C. to about 70° C. In some examples, an elongation temperature can be from about 45° C. to about 65° C. In some examples, an elongation temperature can be from about 35° C. to about 65° C. In some examples, an elongation temperature can be from about 40° C. to about 60° C. In some examples, an elongation temperature can be from about 50° C. to about 60° C. In still other examples, an elongation temperature can be at least or about 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

Elongation durations can vary depending upon, for example, the particular biological sample analyzed, the particular source of target nucleic acid (e.g., viral particle, bacteria) in the biological sample, the reagents used, and/or the desired reaction conditions. For example, an elongation duration can be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, an elongation duration can be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

In any of the various aspects, multiple cycles of a primer extension reaction can be conducted. Any suitable number of cycles can be conducted. For example, the number of cycles conducted can be less than about 100, 90, 80, 70, 60, 50, 40, 30, 20, 10, or 5 cycles. The number of cycles conducted can depend upon, for example, the number of cycles (e.g., cycle threshold value (Ct)) necessary to obtain a detectable amplified product (e.g., a detectable amount of amplified DNA product that is indicative of the presence of a target RNA in a biological sample). For example, the number of cycles necessary to obtain a detectable amplified product (e.g., a detectable amount of DNA product that is indicative of the presence of a target RNA in a biological sample) can be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target RNA in a biological sample) can be obtained at a cycle threshold value (Ct) of less than 100, 75, 70, 65, 60, 55, 50, 45, 40, 35, 30, 25, 20, 15, 10, or 5.

The time for which amplification yields a detectable amount of amplified product indicative of the presence of a target nucleic acid amplified can vary depending upon the biological sample from which the target nucleic acid was obtained, the particular nucleic acid amplification reactions to be conducted, and the particular number of cycles of amplification reaction desired. For example, amplification of a target nucleic acid can yield a detectable amount of amplified product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a target RNA can yield a detectable amount of amplified DNA product indicative to the presence of the target RNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, a reaction mixture can be subjected to a plurality of series of primer extension reactions. An individual series of the plurality can comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series can differ from another individual series in a plurality of series, for example, with respect to any one, two, three, or all four of denaturing temperature, denaturing duration, elongation temperature, and elongation duration. Moreover, a plurality of series can comprise any number of individual series such as, for example, at least about or about 2, 3, 4, 5, 6, 7, 8, 9, 10, or more individual series.

For example, a plurality of series of primer extension reactions can comprise a first series and a second series. The first series, for example, can comprise more than ten cycles of a primer extension reaction, where each cycle of the first series comprises (i) incubating a reaction mixture at about 87° C. to about 95° C. for no more than 30 seconds followed by (ii) incubating the reaction mixture at about 35° C. to about 65° C. for no more than about one minute. The second series, for example, can comprise more than ten cycles of a primer extension reaction, where each cycle of the second series comprises (i) incubating the reaction mixture at about 87° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions could be used.

In some embodiments, a target nucleic acid can be subjected to a denaturing condition prior to initiation of a primer extension reaction. In the case of a plurality of series of primer extension reactions, the target nucleic acid can be subjected to a denaturing condition prior to executing the plurality of series or can be subjected to a denaturing condition between series of the plurality. For example, the target nucleic acid can be subjected to a denaturing condition between a first series and a second series of a plurality of series. Non-limiting examples of such denaturing conditions include a denaturing temperature profile (e.g., one or more denaturing temperatures) and a denaturing agent.

An advantage of conducting a plurality of series of primer extension reaction can be that, when compared to a single series of primer extension reactions under comparable denaturing and elongation conditions, the plurality of series approach yields a detectable amount of amplified product that is indicative of the presence of a target nucleic acid in a biological sample with a lower cycle threshold value. Use of a plurality of series of primer extension reactions can reduce such cycle threshold values by at least about or about 1%, 5%, 10%, 15%, 20%, 25%, 30%, 35%, 40%, 45%, 50%, 55%, 60%, 65%, 70%, 75%, 80%, 85%, 90%, or 95% when compared to a single series under comparable denaturing and elongation conditions.

In some embodiments, a biological sample can be preheated prior to conducting a primer extension reaction. The temperature (e.g., a preheating temperature) at which and duration (e.g., a preheating duration) for which a biological sample is preheated can vary depending upon, for example, the particular biological sample being analyzed. In some examples, a biological sample can be preheated for no more than about 60 minutes, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 45 seconds, 30 seconds, 20 seconds, 15 seconds, 10 seconds, or 5 seconds. In some examples, a biological sample can be preheated at a temperature from about 80° C. to about 110° C. In some examples, a biological sample can be preheated at a temperature from about 90° C. to about 100° C. In some examples, a biological sample can be preheated at a temperature from about 87° C. to about 95° C. In some examples, a biological sample can be preheated at a temperature from about 90° C. to about 97° C., e.g., from about 92° C. to about 95° C. In some examples, a biological sample can be preheated at a temperature from about 87° C. to about 95° C. In still other examples, a biological sample can be preheated at a temperature of above or about 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

In any of the various aspects, the time required to complete the elements of a method can vary depending upon the particular steps of the method. For example, an amount of time for completing the elements of a method can be from about 5 minutes to about 120 minutes. In other examples, an amount of time for completing the elements of a method can be from about 5 minutes to about 60 minutes. In other examples, an amount of time for completing the elements of a method can be from about 5 minutes to about 30 minutes. In other examples, an amount of time for completing the elements of a method can be less than or equal to 120 minutes, less than or equal to 90 minutes, less than or equal to 75 minutes, less than or equal to 60 minutes, less than or equal to 45 minutes, less than or equal to 40 minutes, less than or equal to 35 minutes, less than or equal to 30 minutes, less than or equal to 25 minutes, less than or equal to 20 minutes, less than or equal to 15 minutes, less than or equal to 10 minutes, or less than or equal to 5 minutes.

Detection of signals from the sample undergoing amplification can occur throughout the process. The detection can occur continuously or at one or more points during the amplification process. The sample can emit optical signals throughout the process. The optical signals can be related to the amount of amplified target nucleic acid in the sample. Signals can be detected by an external detector or by a detector of a device of the present disclosure.

Thermal zones

An apparatus of the present disclosure (e.g., a thermocycler) can be used to conduct one or more methods of the present disclosure. An apparatus of the present disclosure can comprise one or more thermal zones. A thermal zone can be maintained at or about a constant temperature level, such as within about plus or minus 5° C., 4° C., 3° C., 2° C., 1.2° C., 1° C., 0.7° C., 0.5° C., 0.3° C., 0.1° C., 0.05° C., 0.01° C., 0.005° C., or 0.001° C. A temperature level can be a target temperature level, such as a temperature useful for a reaction step. For example, FIG. 2 shows a first process step 202 using a first target temperature level (e.g., 95° C.) and a second process step 204 using a second target temperature level (e.g., 55° C.). A temperature level can be an overshooting temperature level, such as a temperature higher or lower than a target temperature level. For example, a first overshooting temperature level of 135° C. can be used in heating a sample to a first target temperature level of 95° C., and a second overshooting temperature level of 8° C. can be used in cooling a sample to a second target temperature level of 55° C.

An apparatus can comprise multiple thermal zones, held constantly at similar or different temperature levels. For example, an apparatus can comprise four thermal zones including a first target thermal zone, a corresponding first overshooting thermal zone, a second target thermal zone, and a corresponding second overshooting thermal zone. An apparatus can comprise 1, 2, 3, 4, 5, 6, 7, 8, 9, 10, or more thermal zones. An apparatus can comprise 1, 2, 3, 4, 5, 6, 7, 8, 9, 10, or more target thermal zones. An apparatus can comprise 1, 2, 3, 4, 5, 6, 7, 8, 9, 10, or more overshooting thermal zones. One overshooting thermal zone can be used in conjunction with one or more target thermal levels. For example, an overshooting thermal zone at 135° C. can be used to heat a sample to a first target temperature level of 70° C., and can also be used to heat a sample to a second target temperature level of 95° C.

A target thermal zone can be set to a target temperature level. A target temperature level can be from about 80° C. to about 100° C. For example, a target temperature level can be from about 87° C. to about 95° C. A target temperature level can be from about 90° C. to about 95° C. A target temperature level can be from about 92° C. to about 95° C. A target temperature level can be above or about 90° C., 91° C., 92° C., 93° C., 94° C., 95° C., 96° C., 97° C., 98° C., 99° C., or 100° C. A target temperature level can be from about 40° C. to about 70° C. A target temperature level can be from about 50° C. to about 60° C. A target temperature level can be above or about 40° C., 45° C., 50° C., 51° C., 52° C., 53° C., 54° C., 55° C., 56° C., 57° C., 58° C., 59° C., 60° C., 65° C., 70° C., 75° C., 80° C., or 85° C.

An overshooting thermal zone can be set to an overshooting temperature level. An overshooting temperature level can be from about 110° C. to about 140° C. An overshooting temperature level can be from about 125° C. to about 135° C. An overshooting temperature level can be above or about 110° C., 115° C., 120° C., 125° C., 126° C., 127° C., 128° C., 129° C., 130° C., 131° C., 132° C., 133° C., 134° C., 135° C., 136° C., 137° C., 138° C., 139° C., 140° C., 145° C., or 150° C. An overshooting temperature level can be from about 0° C. to about 35° C., e.g., from about 0° C. to about 30° C. An overshooting temperature level can be from about 0° C. to about 20° C. An overshooting temperature level can be from about 5° C. to about 10° C. An overshooting temperature level can be above or about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., or 35° C. In some embodiments, an overshooting thermal zone can also be employed to maintain a sample volume at a first target temperature level and/or a second target temperature level. This can be achieved, for example, by making the overshooting thermal zone and the sample volume to switch (such as quickly switching) alternately between the states of in and out of thermal contact with each other. In some embodiments, the overshooting thermal zone can comprise a first heating module and a second heating module, which are driven to switch between an open position and a closed position, thereby switching alternately between the states of in and out of thermal contact with the sample volume.

In some cases, an apparatus can comprise a first target thermal zone held at a first target temperature level from about 92° C. to about 95° C., a first overshooting thermal zone held at a first overshooting temperature level from about 110° C. to about 140° C., a second target thermal zone held at a second target temperature level from about 40° C. to about 70° C., and a second overshooting thermal zone held at a second overshooting temperature level from about 0° C. to about 20° C. In some cases, an apparatus can comprise a first target thermal zone held at a first target temperature level of about 95° C., a first overshooting thermal zone held at a first overshooting temperature level of about 135° C., a second target thermal zone held at a second target temperature level of about 55° C., and a second overshooting thermal zone held at a second overshooting temperature level of about 8° C.

A thermal zone can remain set to a particular temperature level throughout an entire thermal cycling operation. Alternatively, a thermal zone can be changed from one temperature level to another during a thermal cycling operation. A thermal zone can be set to 1, 2, 3, 4, 5, or more different temperature levels during a thermal cycling operation.

An apparatus can utilize conduction, convection, radiation, or combinations thereof to heat and/or cool the samples. Thermal zones can comprise heating blocks, heating modules and/or cooling modules. For example, a heating block can be provided that can directly contact the sample, or can contact a sample container that contains the sample, thereby being in thermal contact with the sample. Heat blocks can be used, including but not limited to liquid metal heat blocks and solid metal heat blocks. A heating system using thermally conductive fluid can be used. Alternatively, no thermally conductive fluid can be used. In some instances, a high density of heating and/or cooling elements can be provided for a heat block. In some instances, electricity can be used to resistively heat a heating/cooling system of the thermal cycler. Other techniques, such as induction heating can be used to control the heating/cooling system of the thermal cycler. In some instances Peltier devices can be used to heat or cool the samples in the thermal cycler.

An apparatus can comprise thermal insulation between thermal zones. Thermal insulation can be used to prevent or reduce thermal conduction, such as thermal conduction between thermal zones or between a thermal zone and a reaction vessel. Exemplary thermal insulation materials include but are not limited to air or other gases, vacuum, foam, plastic, glass, rubber, textiles (e.g., paper, wool), fiberglass, or other thermal insulators.

Thermal zones can comprise indentations, slots, holes, depressions, or other shapes designed to mate with sample holders. Such designs can provide improved thermal contact between the thermal zone and the sample holder. In other cases, the thermal zones can be flat. In some cases, sample holders can comprise a flat surface to contact a flat surface of a thermal zone.

Thermal zones can be brought into thermal contact with sample volumes (e.g., reaction vessels) through a variety of motions. Thermal zones can be moved into contact with sample volumes, or sample volumes can be moved into contact with thermal zones. In some cases, thermal zones can be mounted on or otherwise coupled to moveable elements, such as arms (e.g. linear arms, rotating arms), belts, cams, discs, levers, tracks, or wheels. Such moveable elements can be driven by one or more motors, springs, or other driving elements. In some cases, sample volumes (e.g., in sample holders or reaction vessels) can be mounted on or otherwise coupled to moveable elements, such as arms (e.g. linear arms, rotating arms), belts, cams, discs, levers, tracks, or wheels. Such moveable elements can be driven by one or more motors, springs, or other driving elements. Moveable elements can be coupled or linked to coordinate movement. Movement of moveable elements can be controlled by a timing control system, such as those discussed in this disclosure.

Movements can follow any sort of path, including but not limited to linear, curved, and sinusoidal. In some examples, a curving path for a movement can provide simpler and faster actuation than that provided by a linear motion. In some examples, a curving path for movement can reduce or eliminate the need for high-precision control. In some examples, a curving path for movement can reduce the required volume of the device. In some cases, the sample holder remains stationary while thermal zones move in and out of thermal contact with the sample holder. In some cases, the thermal zones remain stationary while the sample holder moves in and out of thermal contact with the thermal zones. In some cases, both the sample holder and the thermal zones move to bring the sample holder into or out of thermal contact with one or more thermal zones.

The sample holder can be configured to move in a direction orthogonal or about orthogonal to a thermal zone. For example, the sample holder can move vertically while the thermal zones move horizontally. The movement of the sample holder and thermal zones can be synchronized so that the sample holder moves away from the thermal zone (e.g., vertically) while the thermal zone moves away from the sample holder (e.g., horizontally).

For example, FIG. 3 shows a schematic of a thermocycler apparatus 300. This exemplary thermocycler comprises a first overshooting thermal zone 301 mounted on a first rotating arm 305, a second overshooting thermal zone 302 mounted on a second rotating arm, a first target thermal zone 303 mounted on a third rotating arm, and a second target thermal zone 304 mounted on a fourth rotating arm. The rotating arms can each comprise a hook 306 and be connected to a synchronous belt 330. The synchronous belt can be driven by a synchronous belt drive motor 331, and can also drive a heat lid with a sample holder 310 which can hold one or more reaction vessels 312 (e.g., PCR tubes). The thermal zones can comprise tube wells 311 into which reaction vessels can fit for improved thermal contact. The thermocycler can also comprise an optical module 320 with a detector, and the optical module can be driven by an optical module drive motor 321. FIG. 4 shows an exploded schematic view of the exemplary thermocycler, and FIG. 5 shows a side view schematic of the exemplary thermocycler.

Detectors

A detector of the device can detect a signal during a nucleic acid amplification reaction. The detector can detect the signal without removing the sample from the device. In various aspects, the detector can detect amplified product (e.g., amplified DNA product, amplified RNA product). Detection of amplified product, including amplified DNA, can be accomplished with any suitable detection method. The particular type of detection method used can depend, for example, on the particular amplified product, the type of reaction vessel used for amplification, other reagents in a reaction mixture, whether or not a reporter agent was included in a reaction mixture, and if a reporter agent was used, the particular type of reporter agent use. Non-limiting examples of detection methods include optical detection, spectroscopic detection, electrostatic detection, and electrochemical detection. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include but are not limited to gel based techniques, such as, for example, gel electrophoresis. Electrochemical detection methods include, but are not limited to, electrochemical detection of amplified product after high-performance liquid chromatography separation of the amplified products.

The detector can be mounted on a moveable element, such as those described in this disclosure. The detector can be driven by a separate drive motor (e.g., 321 in FIG. 3) or can be driven by a motor, belt, or other driving element shared with other moveable elements.

The detector can comprise an image sensor or image sensors. The image sensor can be capable of optical detection. The image sensor can comprise a charge-coupled device (CCD) sensor, including a cooled CCD. The image sensor can comprise an active-pixel sensor (APS), such as a CMOS or NMOS sensor. The detector can comprise a laser sensor. The detector can comprise a photodiode, such as an avalanche photodiode. The detector can comprise a photomultiplier tube (PMT). The sensors can comprise a single sensor or multiple sensors, of the same type or of different types.

The detector can detect an optical signal from the sample. The optical signal can be a fluorescent or other luminescent signal from the sample. The optical signal can be generated by the sample in response to a stimulation light provided to the sample. Stimulation light can be provided by a light source. The light source can comprise a lamp, such as an incandescent, halogen, fluorescent, gas-discharge, arc, or LED lamp. The light source can comprise a laser. The light source can produce a specific wavelength or range or wavelengths, such as UV. The light source can comprise filters for controlling the output wavelength or wavelengths. The light source can comprise multiple light sources, of the same or of different types, which can be used separately or in combination. The light source can be within the device. In some instances, light can be absorbed by the sample, and the sample can emit light. The emitted light can be at the same or different wavelength from the emitted light. In some instances, the optical signal can be a reflection of light from the light source. Alternatively, light can be shined through the sample, and the detector can be capable of detecting the light that passes through the sample.

An optical path can be provided between the sample and the detector. A signal from the sample can reach the detector via the optical path. An optical signal from a sample can traverse the optical path to reach the detector. The optical path can include direct line-of-sight between the sample and the detector. In some instances, one or more optical elements can be provided between the sample and the detector. Examples of optical elements can include lenses, mirrors, prisms, diffusers, concentrators, filters, dichroics, optical fibers, or any other type of optical elements. The optical path may be provided entirely within a housing of the device. The housing can optically isolate the optical path from the surrounding environment. For example, the housing can be light-tight so that little or no interfering optical signals can be provided within the housing that can interfere with the optical path. Light from outside the housing can be blocked from entering the interior of the housing. This can advantageously reduce inaccuracies in the optical signal detected by the detector. The optical path can remain while the nucleic acid amplification is occurring. The detector can be able to continuously or periodically detect signals from the ample while the nucleic acid amplification is occurring via the optical path.

In some embodiments, information regarding the presence of and/or an amount of amplified product (e.g., amplified DNA product) can be outputted to a recipient. Information regarding amplified product can be outputted via any suitable approach. Such information can be provided in real-time while the nucleic-acid amplification is underway. In other instances, the information can be provided once the nucleic acid amplification has been completed. In some instances, some data can be provided in real-time while other data can be presented once the amplification is completed.

In some embodiments, such information can be provided verbally to a recipient. In some embodiments, such information can be provided in a report. A report can include any number of desired elements, with non-limiting examples that include information regarding the subject (e.g., sex, age, race, health status, etc.) raw data, processed data (e.g. graphical displays (e.g., figures, charts, data tables, data summaries), determined cycle threshold values, calculation of starting amount of target polynucleotide), conclusions about the presence of the target nucleic acid, diagnosis information, prognosis information, disease information, and the like, and combinations thereof. The report can be provided as a printed report (e.g., a hard copy) or can be provided as an electronic report. In some embodiments, including cases where an electronic report is provided, such information can be outputted via an electronic display, such as a monitor or television, a screen operatively linked with a unit used to obtain the amplified product, a tablet computer screen, a mobile device screen, and the like. Both printed and electronic reports can be stored in files or in databases, respectively, such that they are accessible for comparison with future reports.

Moreover, a report can be transmitted to the recipient at a local or remote location using any suitable communication medium including, for example, a network connection, a wireless connection, or an internet connection. In some embodiments, a report can be sent to a recipient's device, such as a personal computer, phone, tablet, or other device. The report can be viewed online, saved on the recipient's device, or printed. A report can also be transmitted by any other suitable approach for transmitting information, with non-limiting examples that include mailing a hard-copy report for reception and/or for review by a recipient.

Moreover, such information can be outputted to various types of recipients. Non-limiting examples of such recipients include the subject from which the biological sample was obtained, a physician, a physician treating the subject, a clinical monitor for a clinical trial, a nurse, a researcher, a laboratory technician, a representative of a pharmaceutical company, a health care company, a biotechnology company, a hospital, a human aid organization, a health care manager, an electronic system (e.g., one or more computers and/or one or more computer servers storing, for example, a subject's medical records), a public health worker, other medical personnel, and other medical facilities.

Power Unit

In some instances, a low voltage can be used to power the device. For example, 12 V or less can be used to power the device. The low voltage can be used to power the detector and the thermal cycler. A low voltage can be used for thermocycling. In some embodiments, the low voltage can be less than or equal to about 60 V, 50 V, 48 V, 40 V, 30 V, 24 V, 20 V, 18 V, 16 V, 15 V, 14 V, 13 V, 12V, 11 V, 10V, 9 V, 8V, 7 V, 6 V, 5 V, 4 V, 3 V, 2 V, or 1 V to perform the thermal cycling. In some instances, the a low voltage of less than or equal to about 50 V, 40 V, 30 V, 24 V, 20 V, 18 V, 16 V, 15 V, 14 V, 13 V, 12V, 11 V, 10V, 9 V, 8V, 7 V, 6 V, 5 V, 4 V, 3 V, 2 V, or 1 V can be used to perform the combination of thermal cycling and detecting.

In some instances, a low degree of power can be used for thermal cycling, or the combination of thermal cycling and detecting. For instance, about 84 W can be used to perform the thermal cycling and detecting. In some instances, a low power can be less than or equal to about 250 W, 200 W, 150 W, 130 W, 120 W, 110 W, 100 W, 90 W, 85 W, 84 W, 83 W, 80 W, 75 W, 70 W, 65 W, 60 W, 55 W, 50 W, 45 W, 40 W, 35 W, 30 W, 25 W, 20 W, 15 W, 10 W, 5 W, 1 W, 500 mW, 100 mW, 50 mW, 10 mW, 5 mW, or 1 mW. The amount of power used to operate the device can be less than or equal to any of the values described herein. Alternatively, the amount of power used to operate the device can be greater than equal to any of the values described herein. The amount of power used to operate the device can fall into a range between any two of the values described herein. The amount of power used to operate the thermal cycler and detector can have a total less than any of the values described herein. The amount of power used to operate the thermal cycler and detector can have a total greater than any of the values described herein. The amount of power used to operate the thermal cycler and detector can fall into a range between any two of the values described herein.

The device can also be operably linked to an energy storage device. The energy storage device can be a battery pack. The battery pack can be a portable battery pack. The battery pack can comprise one or more batteries. The batteries can be an electrochemical energy storage device. For example, the battery pack can include a single or multiple battery cells. The battery can be a lithium-based battery, such as a lithium ion battery. The battery can have any chemistry, including but not limited to lead acid batteries, valve regulated lead acid batteries (e.g., gel batteries, absorbed glass mat batteries), nickel-cadmium (NiCd) batteries, nickel-zinc (NiZn) batteries, nickel metal hydride (NIMH) batteries, or lithium-ion (Li-ion) batteries.

The energy storage device can be part of the device. In one example, the energy storage device can be provided within a housing of the device. The energy storage device can be removable from the device or can be an integral part of the device. In some instances, the energy storage device can be placed within the housing of the device and/or removed from within the housing of the device. Energy storage devices can be swapped or exchanged. In some instances, the energy storage devices can be rechargeable. The energy storage devices can be rechargeable while within the device, or can be removed to be recharged.

In another example, the energy storage device can be directly attached to the device but not within the housing of the device. For example, an external attachment and/or connection can be provided. The energy storage device can directly contact the device housing. The energy storage device can be attached to the device and into place via one or more connector or mechanical fastener. The energy storage device can be separably attached to the device. For example, the energy storage can be attached and detached from the device. Energy storage devices can be swapped. The energy storage device can be rechargeable. The energy storage devices can be rechargeable while attached to the device, or can be separated to be recharged.

The energy storage device can be electrically connected to the device via one or more connector. For example, the connector can be a wire, cable, or other conductive pathway. The connector may be a flexible conductive pathway. For example, the energy storage device can be plugged into the device or vice versa. The energy storage device and the device can be separable from one another. Different energy storage devices can be swapped for the device. For example, the device can plug into different energy storage devices. The energy storage device can be rechargeable. The energy storage devices can be rechargeable while electrically connected to the device, or can be separated to be recharged. A physical electrical connection can be provided between the energy storage device and the device. Alternatively, the energy storage device can wirelessly power the device.

The energy storage device can use low voltage to power the device. For example, the energy storage device can provide no more than 12 V or other voltage values described elsewhere herein to power the device. The storage device can use no more than a total of 12 V (or any other voltage value described elsewhere herein) to power the thermal cycler and the detector of the device. Other components of the device (e.g., input module, output module, light source, or processors) may also be powered using no more than a total of 12 V.

The energy storage device can receive a low voltage power when charging the device. For example, no more than 12 V, or other voltage values described elsewhere herein, can be used to charge the energy storage device. The energy storage device can output energy at the same voltage as it receives.

In some instances, when energy is coming in from an external power source, the device can be powered directly from the external power source. In another example, even when energy is coming in from an external power source, the device can be powered through the energy storage device, and the external power source can be used to charge the energy storage device. In some instances, the energy coming in from the external power source can be used to power the device when the energy storage unit is fully charged.

As previously described any low voltage power can be used to power the device Similarly, any low voltage power can be used to charge the energy storage device. Any reference to low voltage can include a voltage of 50 V or less, 40 V, or less, 35 V, or less, 30 V, or less, 25 V or less, 24 V or less, 22 V or less, 20 V or less, 19 V or less, 18 V or less, 17 V or less, 16 V or less, 15 V or less, 14 V or less, 13.5 V or less, 13 V or les, 12.5 V or less, 12 V or less, 11.5 V or less, 11 V or less, 10.5 V or less, 10 V or less, 9.5 V or less, 9 V or less, 8 V or less, 7 V or less, 6 V or less, 5 V or less, 4 V or less, 3 V or less, 2 V or less, 1 V or less, 500 mV or less, 200 mV or les, 100 mV or less, 50 mV or less, 10 mV or less, 5 mV or less, or 1 mV or less.

The device can be capable of operating at low power. Any combination of components can be capable of operating at low power. For example, the thermal cycler and the detector can be capable of operating at a combined low power. The thermal cycler and detector and input unit can be capable of operating at a combined low power. The thermal cycler, detector, input unit and output unit can be capable of operating at a combined low power. Any reference to a low power can include a power of 250 W or less, 200 W or less, 150 W or less, 130 W or less, 120 W or less, 110 W or less, 100 W or less, 90 W or less, 85 W or less, 84 W or less, 83 W or less, 80 W or less, 75 W or less, 70 W or less, 65 W or less, 60 W or less, 55 W or less, 50 W or less, 45 W or less, 40 W or less, 35 W or less, 30 W or less, 25 W or less, 20 W or less, 15 W or less, 10 W or less, 5 W or less, 1 W or less, 500 mW or less, 100 mW or less, 50 mW or less, 10 mW or less, 5 mW or less, 1 mW or less, or any other power value described elsewhere herein.

Any description of a battery pack can apply to any other type of energy storage device and vice versa. The battery pack can receive a low voltage input. For example, the low voltage input can be 12 V or less, or any other voltage described elsewhere herein. The voltage input can be provided from an external power source. In some instances, the external power source can be a charging port in a vehicle or a facility. For example, an electrical outlet or other type of charging port can be used. In another example, the external power source can be a power generation device. In some instances, the power generation device can provide power by use of kinetic energy (e.g., crank or dynamo), renewable energy source (e.g., solar, wind, water, geothermal), chemical, nuclear, or any other type of power generation source. External power sources can include on-grid or off-grid power sources. The voltage input can be direct current (DC) and/or alternating current (AC).

The voltage input can be provided to a charging circuit. The charging circuit can be in electrical communication with a current protection circuit and a battery. The charging circuit and/or current protection circuit can prevent overcharging of the battery. For example, overvoltage can be prevented. The charging circuit and/or current protection circuit can regulate charging of the battery. A single battery or multiple batteries can be provided in a battery pack. If multiple batteries are provided, they can be connected in series, in parallel or any combination thereof.

The current protection circuit and battery can be coupled to a boost converter and/or voltage regulator. In one example, the boost converter can include a voltage-step-up. The voltage-step-up can be DC-DC. The voltage regulator can control the battery pack to maintain constant voltage. For instance, the boost converter and voltage regulator can permit the voltage output from the battery pack to remain constant. The voltage output can be a low voltage, such as 12 V or less, or any other voltage value described elsewhere herein.

In some embodiments, the voltage input can equal the voltage output. The voltage input may or may not be constant. The voltage output can remain constant. The voltage output can be a voltage used to power a device. The voltage output can be DC.

The output from the battery can be at any current. In some examples, the output can be at 7 amps. The current value may be a maximum current value. Any other embodiments, any current value may be provided, such as about 50 A or less, 30 A or less, 20 A or less, 15 A or less, 13 A or less, 12 A or less, 11 A or less, 10 A or less, 9 A or less, 8 A or less, 7 A or less, 6 A or less, 5 A or less, 4 A or less, 3 A or less, 2 A or less, 1 A or less, 500 mA or less, 200 mA or less, 100 mA or less, 50 mA or less, 10 mA or less, 5 mA or less, or 1 mA or less. In one instance, the output may be 12 V DC with a maximum of 7 A.

The charger power can be at 12 V 7 A DC. The charger power can be at 12 V 10 A DC. In some instances, charger power can be less than or equal to about 84 W. In some instances, the charger power can be less than or equal to about 200 W, 150 W, 120 W, 100 W, 90 W, 88 W, 85 W, 84 W, 83 W, 82 W, 80 W, 75 W, 70 W, 65 W, 60 W, 55 W, 50 w, 45 W, 40 W, 35 W, 30 W, 25 W, 20 W, 15 W, 10 W, 5 W, 3 W, 2 W, 1 W, 500 mw, 100 mW, 50 mW, 10 mW, 5 mW, or 1 mW.

The battery pack can have any capacity. For example, the capacity can be about 13.2 Ah. In other instances, the capacity can be less than or equal to about 100 Ah, 50 Ah, 30 Ah, 25 Ah, 20 Ah, 17 Ah, 16 Ah, 15 Ah, 14 Ah, 13.5 Ah, 13 Ah, 12.5 Ah, 12 Ah, 11 Ah, 10 Ah, 9 Ah, 8 Ah, 7 Ah, 6 Ah, 5 Ah, 4 Ah, 3 Ah, 2 Ah, or 1 Ah.

The battery pack can require any amount of time to become fully charged. In one example, the charge time (e.g., from empty to fully charged) can be about 5 hours. In some instances, the charging time can be less than or equal to about 20 hours, 15 hours, 12 hours, 10 hours, 8 hours, 7 hours, 6.5 hours, 6 hours, 5.5 hours, 5 hours, 4.5 hours, 4 hours, 3.5 hours, 3 hours, 2 hours, 1 hour, 45 minutes, 30 minutes, 20 minutes, 15 minutes, 10 minutes, 8 minutes, 7 minutes, 6 minutes, 5 minutes, 3 minutes, 2 minutes, 1 minute, 45 seconds, 30 seconds, 15 seconds, or 10 seconds. In some instances, the charging time can be greater than or equal to any of the charge times described herein. The charging time can fall within a range between any two of the values described herein.

The battery pack can have any working duration. The working duration can include the amount of time the battery pack can operate from a fully charged state to a fully discharged state. In some instances, the working duration can be less than the charging time. Alternatively, the working duration can be greater than or equal to the charging time. The working duration can be about 4 hours or less. In some instances, the working duration can be less than or equal to about 20 hours, 15 hours, 12 hours, 10 hours, 8 hours, 7 hours, 6 hours, 5 hours, 4.5 hours, 4 hours, 3.5 hours, 3 hours, 2.5 hours, 2 hours, 1.5 hours, 1 hour, 45 minutes, 30 minutes, 20 minutes, 15 minutes, 10 minutes, 8 minutes, 7 minutes, 6 minutes, 5 minutes, 3 minutes, 2 minutes, 1 minute, 45 seconds, 30 seconds, 15 seconds, or 10 seconds. In some instances, the working durations can be greater than or equal to any of the working durations described herein. The working durations can fall within a range between any two of the values described herein.

Any dimensions can be provided for a battery pack. The batter pack can be portable. The battery pack can be capable of being lifted and carried by a human. The battery pack can be capable of placing in a car. The battery pack can have a maximum dimension (e.g., length, width, height, diagonal, diameter) of no more than about 200 mm. The battery pack can have a maximum dimension of no more than about 1 mm, 3 mm, 5 mm, 7 m, 10 mm, 15 mm, 20 mm, 30 mm, 40 mm, 50 mm, 60 mm, 70 mm, 100 mm, 120 mm, 150 mm, 170 mm, 180 mm, 190 mm, 200 mm, 210 mm, 220 mm, 250 mm, 270 mm, 300 mm, 350 mm, 400 mm, 450 mm, 500 mm, 550 mm, 600 mm, 700 mm, or 1 m. Alternatively, the battery pack can have a maximum dimension greater than any of the dimension values described herein. In some instances, the battery pack can have a maximum dimension falling within a range between any two of the values described herein.

Any footprint can be provided for the battery pack. The footprint can include a lateral cross-sectional area of the battery pack. The footprint can include an area of a surface that the battery pack would occupy when resting on the surface. In some instances, the battery pack can have a footprint of less than or equal to about 1 cm², 5 cm², 10 cm², 15 cm², 20 cm², 25 cm², 30 cm², 40 cm², 50 cm², 60 cm², 70 cm², 80 cm², 90 cm², 100 cm², 120 cm², 150 cm², 200 cm², 250 cm², 300 cm², 350 cm², 400 cm², 500 cm², 600 cm², 700 cm², 800 cm², 900 cm², 1000 cm², 1200 cm², 1500 cm², 1700 cm², or 2000 cm². The battery pack can have a footprint greater than or equal to any of the values described herein. The battery pack can have a footprint falling into a range between any two of the values described herein.

The battery pack can have any volume. In some instances, the battery pack can have the dimensions of about 200 mm×200 mm×50 mm. The battery pack can have a volume of about 2000 cm3. In some instances, the battery can have a volume of less than about 1 cm³, 5 cm³, 10 cm³, 15 cm³, 20 cm³, 25 cm³, 30 cm³, 40 cm³, 50 cm³, 60 cm³, 70 cm³, 80 cm³, 90 cm³, 100 cm³, 120 cm³, 150 cm³, 200 cm³, 250 cm³, 300 cm³, 350 cm³, 400 cm³, 500 cm³, 600 cm³, 700 cm³, 800 cm³, 900 cm³, 1000 cm³, 1200 cm³, 1500 cm³, 1700 cm³, 2000 cm³, 2200 cm³, 2500 cm³, 3000 cm³, 3500 cm³, 4000 cm³, 5000 cm³, 7000 cm³, or 10,000 cm³. The battery pack can have a volume greater than any of the volumes described herein. The battery pack can have a volume falling within a range between any two of the values described herein.

The battery pack can have any weight. For example, the battery pack can weigh less than or equal to about 1.65 kg. The battery pack can weigh less than or equal to about 1 mg, 10 mg, 100 mg, 1 g, 10 g, 100 g, 200 g, 300 g, 400 g, 500 g, 600 g, 700 g, 800 g, 900 g, 1 kg, 1.1 kg, 1.2, kg, 1.3 kg, 1.4 kg, 1.45 kg, 1.5 kg, 1.55 kg, 1.6 kg, 1.65 kg, 1.7 kg, 1.75 kg, 1.8 kg, 1.85 kg, 1.9 kg, 2 kg, 2.2 kg, 2.5 kg, 3 kg, 3.5 kg, 4 kg, 4.5 kg, 5 kg, 6 kg, 7 kg, 8 kg, 9 kg, or 10 kg. The battery pack can weigh more than any of the values described herein. The battery pack can have a weight falling within a range between any two of the values described herein.

Any of the dimensions or characteristics of the battery pack as described herein can be provided separately or in combination with one another. For example, any of the dimensions, footprints, volumes, and/or weights can be combined with one another and/or with any voltage, current, power, capacity, charging time and/or working duration described herein. The battery pack can have any characteristics described herein while being configured to deliver power to a device for conducting nucleic acid amplification having any of the characteristics and/or components described herein, alone or in combination.

Housing

The thermocycler and/or detector can be housed in a housing. The housing can partially or completely enclose components of the device. The housing can surround components of the device laterally and/or on the top and bottom. The housing can be a rigid structure. For example, the housing can contain the thermal cycler therein. The detector can also be contained within the housing. In other implementations, the detector can be outside the housing of the device. The detector can be an integral part of the device. Alternatively, the detector can be removable or separable from the device.

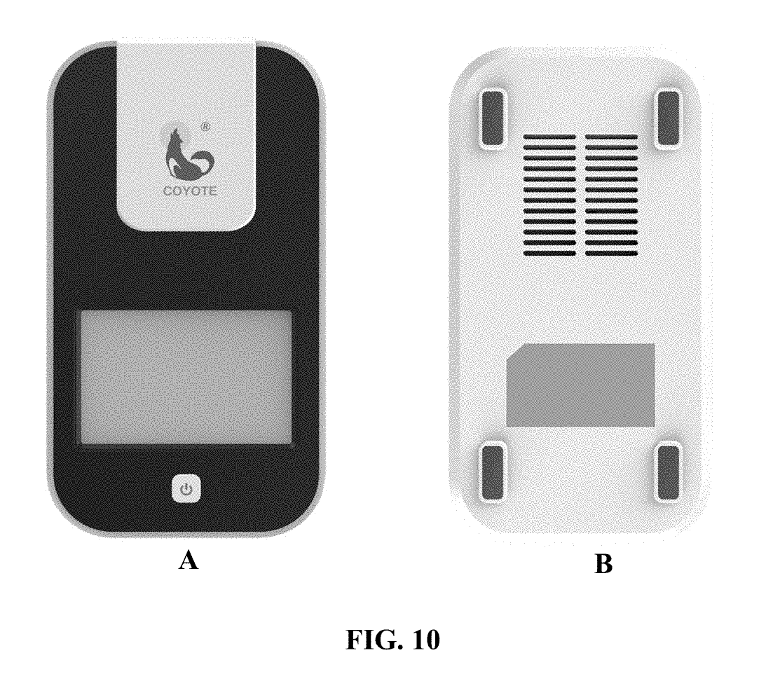

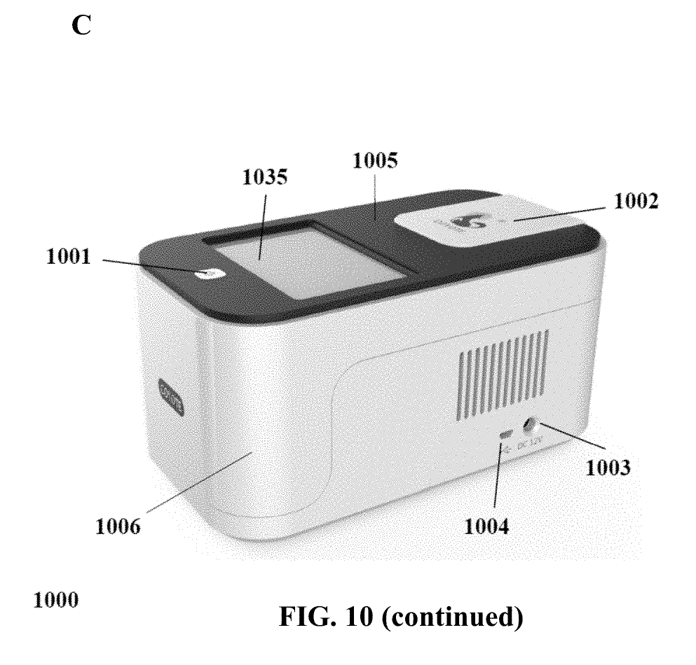

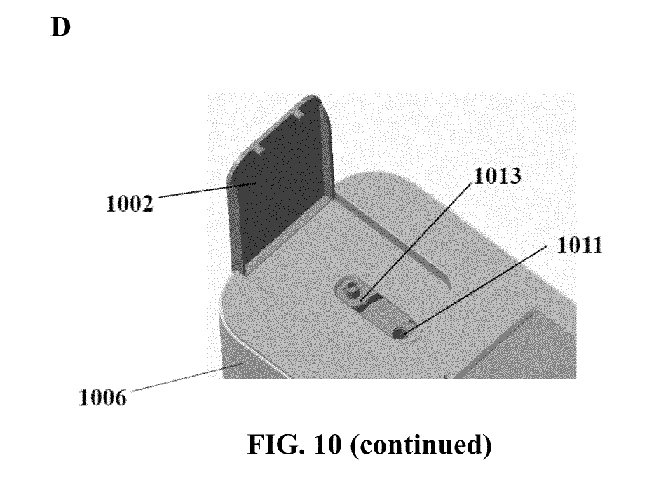

An example of a housing of a thermocycler is shown in FIG. 10. FIG.10 (panel A) shows an exemplary top view of the external enclosure of a housing and FIG. 10 (panel B) shows an exemplary bottom view of the external enclosure of a housing. FIG. 10 (panel C) shows an exemplary stereo view of a housing 1000 of an exemplary thermocycler comprising a control panel 1005 on top of a base case 1006, wherein the control panel 1005 can comprise a switch button 1001, an electronic display 1035 and a lid 1002, and wherein the base case 1006 can comprise a power port 1003 and a USB connector 1004. FIG. 10 (panel D) shows an exemplary enlarged partial view of the top of a housing with the lid 1002 open, revealing a sample rack 1013 and a tube well 1011 incorporated on the upper part of the base case 1006 which may be covered when the lid 1002 is closed.

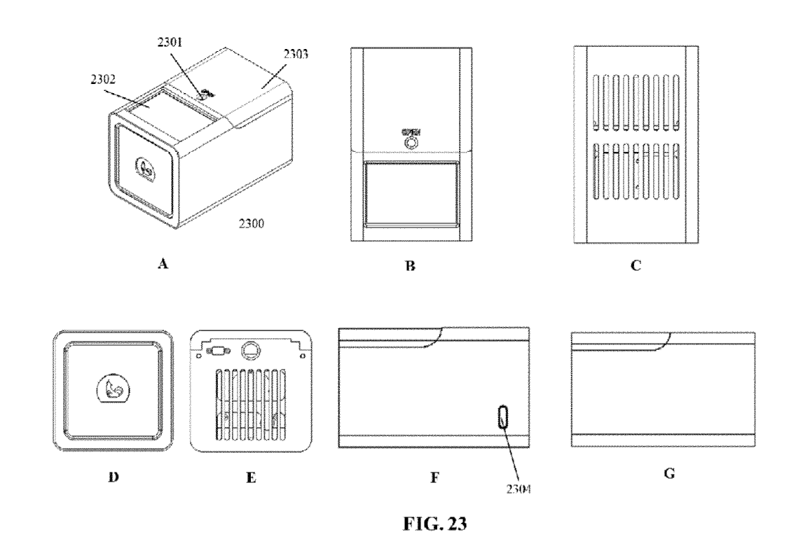

Another example of the housing is provided in FIG. 23. FIG. 23 (panel A) shows a perspective side view of an exemplary thermocycler comprising a switch button 2301, an electronic display 2302 and a lid 2303 on the top side. FIG. 23 (panel B) and FIG. 23 (panel C) show a top view and a bottom view, respectively. FIG. 23 (panel D) and 23 (panel E) show a front view and a back view, respectively, and FIG. 23 (panel F) and 23 (panel G) show a left side view and a right side view, respectively.

As can be seen from FIG. 23 (panel F), on the left side of the housing, there may be a scanning element 2304 capable of scanning, detecting or communication with an identification unit that uniquely identifies the sample being processed and/or the reaction to be performed, thereby obtaining information regarding the sample (e.g., source of the sample, viruses/bacterium suspected to be comprised in the sample, etc.) and/or executing a predetermined protocol of thermal cycling to amplify a particular target nucleic acid. The identification unit may be an identification number or barcode. As an alternative, the identification unit may be a radiofrequency identification (RFID) unit providing a unique or identifiable RFID. The identification unit may be attached to an external surface of a reaction vessel (e.g., a tube), or comprised in the sample to be analyzed.

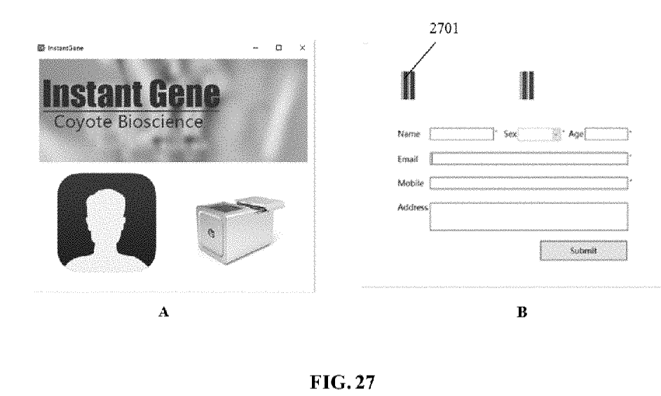

The same identification unit (e.g., the same identification number or barcode) may be scanned by a user (e.g., a user in a remote location, for example, away from the thermocycler) with an electronic device (e.g., a cell phone). After scanning and information processing, a user interface may appear in the electronic device or a computer operatively connected to the electronic device, as shown in FIG. 27A and 27B. The user interface may comprise one or more graphical elements, the one or more graphical elements may include the identification number or barcode 2701 comprised by the identification unit. Further graphical elements may be employed by the user to input personal and contact information, such as name, sex, age, email address, telephone number and mailing address, so that a report (e.g., sent by email and/or short messages) containing detailed and/or simplified results of the amplification reaction/detection may be provided to the user.

The device can have a maximum dimension (e.g., length, width, height, diagonal, diameter) of no more than about 15 cm. In some instances, the device can have a housing no more than 10 cm tall. In another example, the device can have a housing no more than 16 cm in length. The device may have a maximum dimension of no more than about 1 mm, 3 mm, 5 mm, 7 m, 10 mm, 12 mm, 15 mm, 17 mm, 20 mm, 25 mm, 30 mm, 40 mm, 50 mm, 60 mm, 70 mm, 75 mm, 80 mm, 85 mm, 90 mm, 97 mm, 100 mm, 105 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 270 mm, 300 mm, 350 mm, 400 mm, 450 mm, 500 mm, 550 mm, 600 mm, 700 mm, or 1 m. Alternatively, the device can have a maximum dimension greater than any of the dimension values described herein. In some instances, the device can have a maximum dimension falling within a range between any two of the values described herein.

Any footprint can be provided for the device. The footprint can include a lateral cross-sectional area of the device. The footprint can include an area of a surface that the device would occupy when resting on the surface. In some instances, the device can have a footprint of less than or equal to about 1 cm², 5 cm², 10 cm², 15 cm², 20 cm², 25 cm², 30 cm², 40 cm², 50 cm², 60 cm², 70 cm², 80 cm², 90 cm², 100 cm², 120 cm², 150 cm², 200 cm², 250 cm², 300 cm², 350 cm², 400 cm², 500 cm², 600 cm², 700 cm², 800 cm², 900 cm², 1000 cm², 1200 cm², 1500 cm², 1700 cm², or 2000 cm². The device can have a footprint greater than or equal to any of the values described herein. The device can have a footprint falling into a range between any two of the values described herein.

The device can have any volume. In some instances, the battery can have a volume of less than about 1 cm³, 5 cm³, 10 cm³, 15 cm³, 20 cm³, 25 cm³, 30 cm³, 40 cm³, 50 cm³, 60 cm³, 70 cm³, 80 cm³, 90 cm³, 100 cm³, 120 cm³, 150 cm³, 200 cm³, 250 cm³, 300 cm³, 350 cm³, 400 cm³, 500 cm³, 600 cm³, 700 cm³, 800 cm³, 900 cm³, 1000 cm³, 1200 cm³, 1500 cm³, 1700 cm³, 2000 cm³, 2200 cm³, 2500 cm³, 3000 cm³, 3500 cm³, 4000 cm³, 4500 cm³, 5000 cm³, 5500 cm³, 6000 cm³, 7000 cm³, 8000 cm³, 9000 cm³, or 10,000 cm³. The device can have a volume greater than any of the volumes described herein. The device can have a volume falling within a range between any two of the values described herein.

The device can have any weight. For example, the device can weigh less than or equal to about 2 kg. The device can weigh less than or equal to about 1 mg, 10 mg, 100 mg, 1 g, 10 g, 100 g, 200 g, 300 g, 400 g, 500 g, 600 g, 700 g, 800 g, 900 g, 1 kg, 1.1 kg, 1.2, kg, 1.3 kg, 1.4 kg, 1.45 kg, 1.5 kg, 1.55 kg, 1.6 kg, 1.65 kg, 1.7 kg, 1.75 kg, 1.8 kg, 1.85 kg, 1.9 kg, 2 kg, 2.1 kg, 2.2 kg, 2.5 kg, 2.7 kg, 3 kg, 3.5 kg, 4 kg, 4.5 kg, 5 kg, 6 kg, 7 kg, 8 kg, 9 kg, or 10 kg. The device can weigh more than any of the values described herein. The device can have a weight falling within a range between any two of the values described herein.

Any of the dimensions or characteristics of the device as described herein can be provided separately or in combination with one another. For example, any of the dimensions, footprints, volumes, and/or weights can be combined with one another and/or with any voltage, current, power, described herein. The device can have any characteristics described herein while being configured to conduct nucleic acid amplification and/or real-time detection of the nucleic acid amplification. The device can be a portable device having any of the dimensions described herein while being able to operate at low voltage power. This can advantageously take full advantage of the device's portability, not only in size but ability to be powered from a wider range of power sources and/or have longer battery life.

### Computer Control Systems

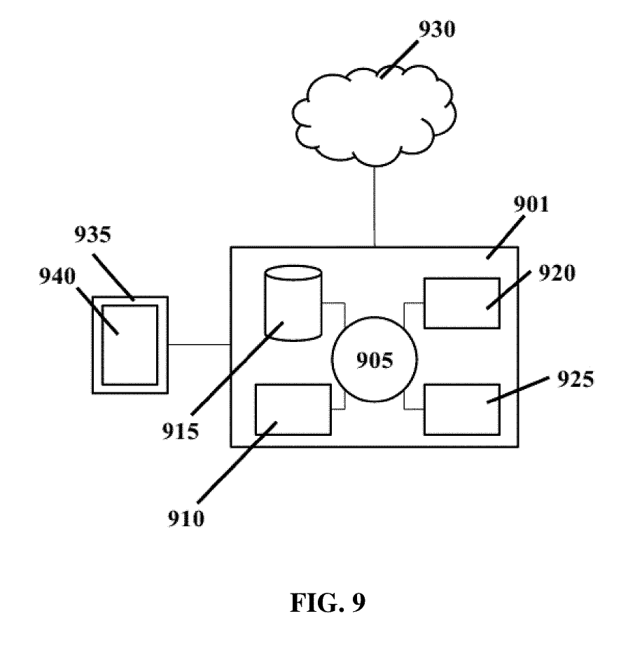

The present disclosure provides computer control systems that are programmed to implement methods of the disclosure. Computer control systems can be configured or integrated within a device (e.g., thermocycler) of the present disclosure. FIG. 9 shows a computer system 901 that is programmed or otherwise configured to control the operation of a thermal cycler and collect data. The computer system 901 can regulate various aspects of thermal cyclers of the present disclosure, such as, for example, target temperature levels, overshooting temperature levels, ramp rates and times, number of cycles, hold times at target temperatures, and data collection. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user (e.g., personal electronic device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, temperature levels, thermal cycling protocol conditions, and signal data from sample volumes. Examples of UI's include, without limitation, a graphical user interface (GUI) and web-based user interface.

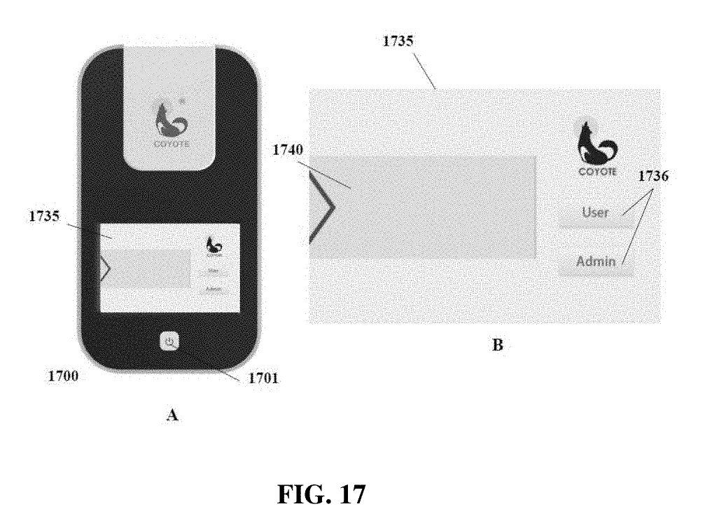

FIG. 17 (panel A) shows an example of a control panel 1700 comprised by an exemplary thermocycler apparatus of the present invention. The control panel 1700 may comprise a switch button 1701 and an electronic display 1735. FIG. 17 (panel B) shows an exemplary electronic display 1735 comprising a user interface (UI) 1740. The electronic display 1735 may also comprise one or more graphical elements 1736 (e.g. such graphical elements may include image and/or textual information, such as pictures, icons and text). The graphical elements can have various sizes and orientations on the user interface. Furthermore, an electronic display screen may be any suitable electronic display including examples described elsewhere herein. Non-limiting examples of electronic display screens include a monitor, a mobile device screen, a laptop computer screen, a television, a portable video game system screen and a calculator screen. In some embodiments, an electronic display screen may include a touch screen (e.g., a capacitive or resistive touch screen) such that graphical elements displayed on a user interface of the electronic display screen can be selected via user touch with the electronic display screen. In an example, the graphical element may be icons demonstrating a user's identity (such as ordinary user or administrator, as illustrated by the icons 1736 shown in FIG. 17 (panel B)).

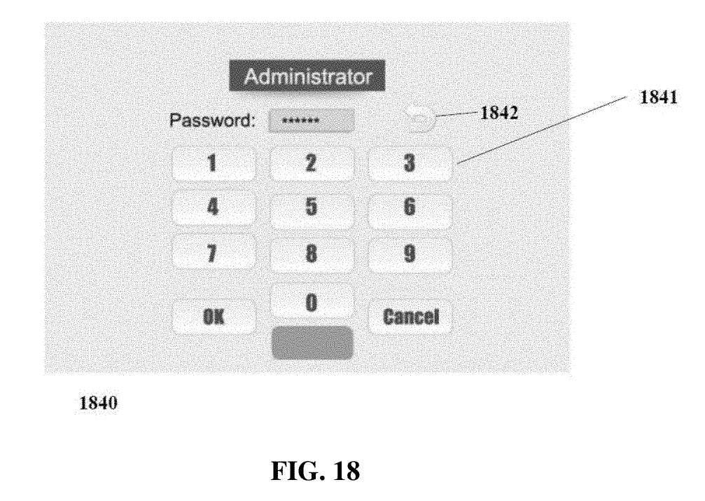

FIG. 18 shows an example user interface 1840 comprising multiple graphical elements, for example, it may comprise an element 1842 accessible by a user to execute a protocol to return to a previous interface. It may also comprise graphical elements 1841 that can be employed by a user to input information, such as a password.

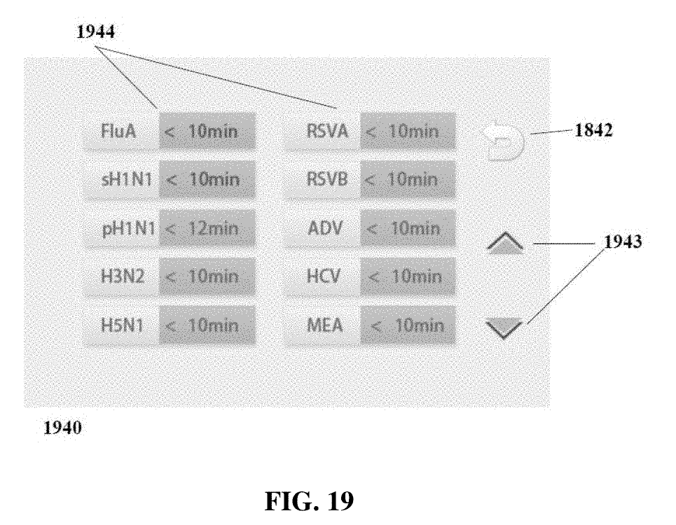

In some embodiments, each of the graphical elements may be associated with a disease or health condition, and a given amplification protocol among the plurality of amplification protocols may be directed to assaying a presence of the disease or health condition in the subject. Thus, in such cases, a user can select a graphical element in order to run an amplification protocol (or series of amplification protocols) to assay for a particular disease or health condition. In some embodiments, the disease or health condition may be associated with a single nucleotide polymorphism (SNP) (e.g., the SNP CYPC2C19). In some embodiments, the disease or health condition may be associated with a virus such as, for example, any RNA virus or DNA virus including examples of such viruses described elsewhere herein. Non-limiting examples of viruses include human immunodeficiency virus I (HIV I), human immunodeficiency virus II (HIV II), an orthomyxovirus, Ebola virus, Dengue virus, influenza viruses (e.g., H1N1 virus, H3N2 virus, H7N9 virus or H5N1 virus), hepevirus, hepatitis A virus, hepatitis B virus, hepatitis C virus (e.g., armored RNA-hepatitis C virus (RNA-HCV)), hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus (e.g., adenovirus type 55 (ADV55), adenovirus type 7 (ADV7)), FluA virus, respiratory syncytial virus A (RSVA), respiratory syncytial virus B (RSVB), measles virus and Varicella virus. In some embodiments, the disease or health condition may be associated with a pathogenic bacterium (e.g., Mycobacterium tuberculosis) or a pathogenic protozoan (e.g., Plasmodium as in Malaria), including examples of such pathogens described elsewhere herein. An example of a user interface having a plurality of graphical elements each associated with a given amplification protocol is shown in FIG. 19. As shown in FIG. 19, an example user interface 1940 includes a display of graphical elements 1944. Each of the graphical elements 1944 can be associated with a particular disease or health condition (e.g., “ADV” adenovirus, “H1N1” for H1N1 virus and “HCV” for Hepatitis C virus) that is, in turn, associated with one or more amplification protocols directed toward the particular disease or health condition. Upon user selection (e.g., user touch when an electronic display screen includes a touch-screen having the user interface 1940) with a particular graphical element, the particular amplification protocol(s) associated with the disease or health condition associated with the graphical element can be executed by an associated computer processor. For example, when a user interacts with graphical element 1944 depicted as “FluA”, one or more amplification protocols associated with assaying for FluA virus can be executed by the associated computer processor. A user interface may have any suitable number of graphical elements each corresponding to a specific disease or health condition. Moreover, where each graphical element shown in the user interface 1940 of FIG. 19 is associated with only one disease or health condition, each graphical element of a user interface can be associated with one or more diseases or health conditions such that an associated computer processor executes a series of amplification protocols (e.g., each individual amplification protocol directed to a particular disease or health condition) upon user selection of the graphical element. For example, a graphical element may correspond to Ebola virus and H1N1 virus such that selection of the graphical element results in an associated computer processor executing amplification protocols for both Ebola virus and H1N1 virus. In addition, the user interface 1940 may also comprise element 1943 accessible by a user to return to a previous page or go to the next page.

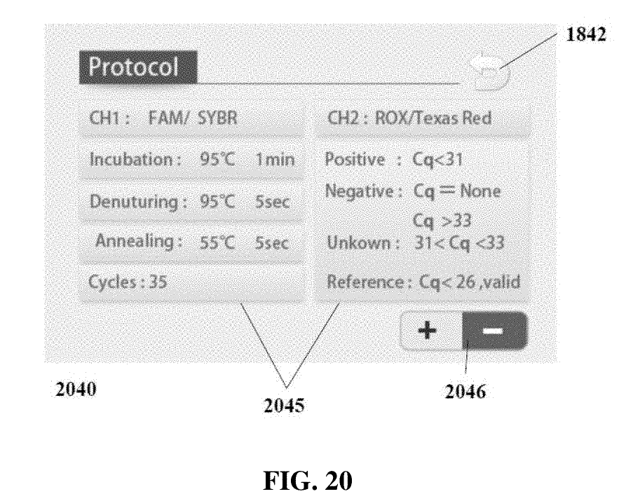

FIG. 20 shows another example user interface 2040 comprising multiple graphical elements. For example, it may comprise element 2045 accessible by a user to adjust specific reaction parameters, such as optical detection channel, incubation temperature, incubation time, denaturing and annealing temperature and time, amplification cycle number and measures of experiment result. It may also comprise a graphical element 2046 that is accessible by a user to change (e.g. increase and/or decrease) certain reaction parameters.

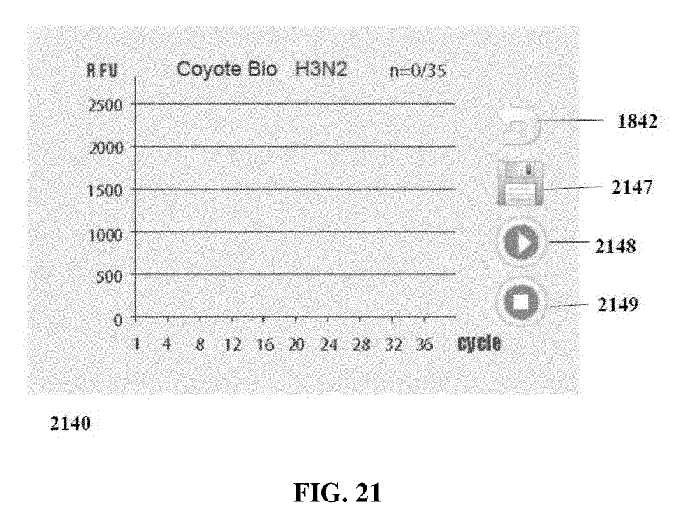

FIG. 21 shows another example user interface 2140 for running an amplification experiment. For example, it may comprise element 2147 accessible by a user to save an experimental result. It may also comprise elements 2148 and 2149 accessible by a user to start and stop an amplification protocol, respectively.

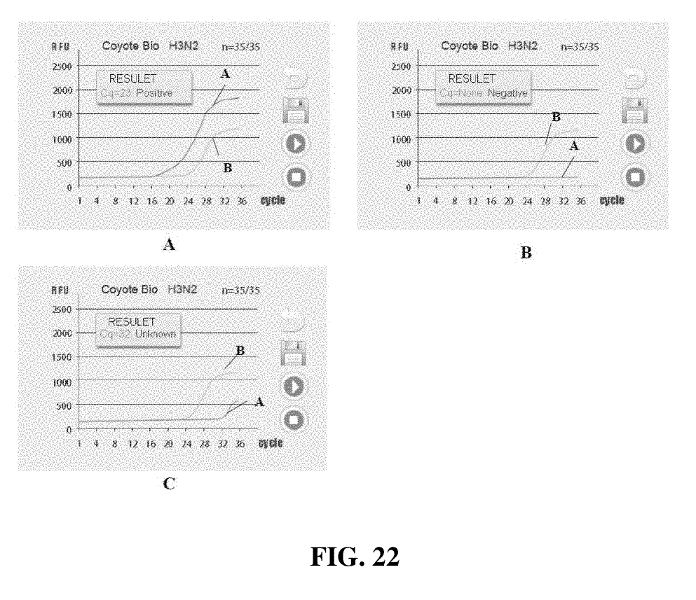

FIG. 22 (panels A-C) provide examples of user interfaces showing graphs depicting results of nucleic acid amplification reactions.

An aspect of the present invention provides a system for amplifying a target nucleic acid in a biological sample obtained from a subject. The system can comprise an electronic display screen that comprises a user interface that displays a graphical element that is accessible by a user to execute an amplification protocol to amplify the target nucleic acid in the biological sample. The system can also comprise a computer processor coupled to the electronic display screen and programmed to execute the amplification protocol upon selection of the graphical element by the user. The amplification protocol can comprise subjecting a reaction mixture comprising the biological sample and reagents necessary for conducting nucleic acid amplification to a plurality of series of primer extension reactions to generate amplified product that is indicative of the presence of the target nucleic acid in the biological sample. Each series of primer extension reactions can include one or more cycles of incubating the reaction mixture under a denaturing condition characterized by a denaturing temperature and a denaturing duration, followed by incubating the reaction mixture under an elongation condition characterized by an elongation temperature and an elongation duration. An individual series may differ from at least one other individual series of the plurality with respect to the denaturing condition and/or the elongation condition.

In some embodiments, the amplification protocol can further comprise selecting a primer set for the target nucleic acid. In some embodiments, the reagents may comprise a deoxyribonucleic acid (DNA) polymerase and a primer set for the target nucleic acid. In some cases, the reagents also comprise a reverse transcriptase. In some embodiments, the user interface can display a plurality of graphical elements. Each of the graphical elements can be associated with a given amplification protocol among a plurality of amplification protocols. In some embodiments, each of the graphical elements may be associated with a disease or health condition. A given amplification protocol among the plurality of amplification protocols can be directed to assaying a presence of the disease or health condition in the subject. In some embodiments, the disease or health condition may be associated with a single nucleotide polymorphism (SNP) (e.g., the SNP CYPC2C19). In some embodiments, the disease or health condition may be associated with a virus such as for example an RNA virus or a DNA virus. In some embodiments, the virus can be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, FluA virus, respiratory syncytial virus A (RSVA), respiratory syncytial virus B (RSVB), measles virus and Varicella virus. In some embodiments, the influenza virus can be selected from the group consisting of H1N1 virus (such as sH1N1 and pH1N1 virus), H3N2 virus, H7N9 virus and H5N1 virus. In some embodiments, the adenovirus may be adenovirus type 55 (ADV55) or adenovirus type 7 (ADV7). In some embodiments, the hepatitis C virus may be armored RNA-hepatitis C virus (RNA-HCV). In some embodiments, the disease or health condition may be associated with a pathogenic bacterium (e.g., Mycobacterium tuberculosis) or a pathogenic protozoan (e.g., Plasmodium).

In some embodiments, the target nucleic acid may be associated with a disease or health condition. In some embodiments, the amplification protocol can be directed to assaying a presence of the disease or health condition based on a presence of the amplified product. In some embodiments, the disease or health condition may be associated with a single nucleotide polymorphism (SNP) (e.g., the SNP CYPC2C19). In some embodiments, the disease or health condition may be associated with a virus such as, for example, an RNA virus or a DNA virus. In some embodiments, the virus can be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, FluA virus, respiratory syncytial virus A (RSVA), respiratory syncytial virus B (RSVB), measles virus and Varicella virus. In some embodiments, the influenza virus can be selected from the group consisting of H1N1 virus, H3N2 virus, H7N9 virus and H5N1 virus. In some embodiments, the adenovirus may be adenovirus type 55 (ADV55) or adenovirus type 7 (ADV7). In some embodiments, the hepatitis C virus may be armored RNA-hepatitis C virus (RNA-HCV). In some embodiments, the disease or health condition may be associated with a pathogenic bacterium (e.g., Mycobacterium tuberculosis) or a pathogenic protozoan (e.g., Plasmodium).

In another aspect of the present invention, it provides a system for amplifying a target nucleic acid present in a biological sample obtained from a subject. The system can comprise an input module that receives a user request to amplify the target nucleic acid in the biological sample. The system can also comprise an amplification module that, in response to the user request receives, in a reaction vessel held by a sample holder, a reaction mixture comprising the biological sample and reagents necessary for conducting nucleic acid amplification, the reagents comprising (i) a DNA polymerase and in some cases a reverse transcriptase, and (ii) a primer set for the target nucleic acid. The amplification module may also subject the reaction mixture in the reaction vessel to a plurality of series of primer extension reactions to generate amplified product that is indicative of the presence of the target nucleic acid in the biological sample. Each series may comprise cycling between at least two target temperature levels for one or more cycles of : (a) placing the sample holder in thermal contact with a first overshooting thermal zone to achieve a first target temperature level; and (b) placing the sample holder in thermal contact with a second overshooting thermal zone to achieve a second target temperature level; wherein the first overshooting thermal zone is at a higher temperature than the first target temperature level, and the second overshooting thermal zone is at a lower temperature than the second target temperature level. In various aspects, each the series may comprise conducting any of the method as described in the present application. The system may further comprise an output module operatively coupled to the amplification module, wherein the output module outputs information regarding the target nucleic acid or the amplified product to a recipient. In certain embodiment, the amplification module may be any of the apparatus as disclosed herein.

In another aspect, the present invention provides a system for amplifying a target nucleic acid in a biological sample obtained from a subject. The system may comprise an electronic display screen comprising a user interface that displays a graphical element that is accessible by a user to execute an amplification protocol to amplify the target nucleic acid in the biological sample. The system can also comprise a computer processor coupled to the electronic display screen and programmed to execute the amplification protocol upon selection of the graphical element by the user. The amplification protocol may be performing any of the method as described in the present application. For example, the amplification protocol can comprise subjecting a reaction mixture comprising the biological sample and reagents necessary for conducting nucleic acid amplification contained in a reaction vessel held by a sample holder to a plurality of series of primer extension reactions to generate amplified product that is indicative of the presence of the target nucleic acid in the biological sample. Each series can comprise cycling between at least two target temperature levels for one or more cycles of : (a) placing the sample holder in thermal contact with a first overshooting thermal zone to achieve a first target temperature level; and (b) placing the sample holder in thermal contact with a second overshooting thermal zone to achieve a second target temperature level; wherein the first overshooting thermal zone is at a higher temperature than the first target temperature level, and the second overshooting thermal zone is at a lower temperature than the second target temperature level.

In some embodiments, the amplification protocol can further comprise selecting a primer set for the target nucleic acid. In some embodiments, the reagents may comprise (i) a deoxyribonucleic acid (DNA) polymerase and in some cases a reverse transcriptase, and (ii) a primer set for the target nucleic acid. In some embodiments, the user interface can display a plurality of graphical elements, wherein each of the graphical elements is associated with a given amplification protocol among a plurality of amplification protocols. In some embodiments, each of the graphical elements can be associated with a disease or health condition, and wherein a given amplification protocol among the plurality of amplification protocols can be directed to assaying a presence of the disease or health condition in the subject.

In some embodiments, the disease or health condition can be associated with a virus, such as an RNA virus and/or DNA virus. In some embodiments, the virus can be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, FluA virus, respiratory syncytial virus A (RSVA), respiratory syncytial virus B (RSVB), measles virus, Varicella virus, H1N1 virus, H3N2 virus, H7N9 virus, H5N1 virus, adenovirus type 55 (ADV55), adenovirus type 7 (ADV7), and armored RNA-hepatitis C virus (RNA-HCV).

In some embodiments, the disease or health condition can be associated with a pathogenic bacterium or a pathogenic protozoan, such as Mycobacterium tuberculosis or Plasmodium.

In various aspects, the system may comprise an input module that receives a user request to amplify a target nucleic acid (e.g., target RNA, target DNA) present in a biological sample obtained directly from a subject. Any suitable module capable of accepting such a user request may be used. The input module may comprise, for example, a device that comprises one or more processors. Non-limiting examples of devices that comprise processors (e.g., computer processors) include a desktop computer, a laptop computer, a tablet computer (e.g., Apple® iPad, Samsung® Galaxy Tab), a cell phone, a smart phone (e.g., Apple® iPhone, Android® enabled phone), a personal digital assistant (PDA), a video-game console, a television, a music playback device (e.g., Apple® iPod), a video playback device, a pager, and a calculator. Processors may be associated with one or more controllers, calculation units, and/or other units of a computer system, or implanted in firmware as desired. If implemented in software, the routines (or programs) may be stored in any computer readable memory such as in RAM, ROM, flash memory, a magnetic disk, a laser disk, or other storage medium. Likewise, this software may be delivered to a device via any known delivery method including, for example, over a communication channel such as a telephone line, the internet, a local intranet, a wireless connection, etc., or via a transportable medium, such as a computer readable disk, flash drive, etc. The various steps may be implemented as various blocks, operations, tools, modules or techniques which, in turn, may be implemented in hardware, firmware, software, or any combination thereof. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

In some embodiments, the input module is configured to receive a user request to perform amplification of the target nucleic acid. The input module may receive the user request directly (e.g. by way of an input device such as a keyboard, mouse, or touch screen operated by the user) or indirectly (e.g. through a wired or wireless connection, including over the internet). Via output electronics, the input module may provide the user's request to the amplification module. In some embodiments, an input module may include a user interface (UI), such as a graphical user interface (GUI), that is configured to enable a user provide a request to amplify the target nucleic acid. A GUI can include textual, graphical and/or audio components. A GUI can be provided on an electronic display, including the display of a device comprising a computer processor. Such a display may include a resistive or capacitive touch screen.

Methods and systems of the present disclosure can be implemented by way of one or more algorithms. An algorithm can be implemented by way of software upon execution by the central processing unit 905. The algorithm can, for example, calculate timing and motions to execute a programmed thermal cycling protocol, or collect and process data from sample volumes (e.g. fluorescent data).

The system may further comprise an identification unit that uniquely identifies the system. The identification unit is detectable by an electronic device of the user. During use, the identification unit may be detected by the electronic device to identify the system, and upon the system being identified, a request to execute the amplification may be directed from the electronic device to the system. The identification unit may be an identification number or barcode. As an alternative, the identification unit may be a radiofrequency identification (RFID) unit providing a unique or identifiable RFID. The identification unit may be attached to an external surface of a reaction vessel (e.g., a tube), comprised in the sample to be analyzed and/or comprised in a receipt provided to the user.

The identification unit (e.g., the identification number or barcode) may be scanned by a user (e.g., a user in a remote location, for example, away from the thermocycler) with an electronic device (e.g., a cell phone). After scanning and information processing, a user interface may appear in the electronic device or a computer operatively connected to the electronic device, as shown in FIG. 27A and 27B. The user interface may comprise one or more graphical elements, the one or more graphical elements may include the identification number or barcode 2701 comprised by the identification unit. Further graphical elements may be employed by the user to input personal and contact information, such as name, sex, age, email address, telephone number and mailing address, so that a report (e.g., sent by email and/or short messages) containing detailed and/or simplified results of the amplification reaction/detection may be provided to the user.

### EXAMPLES

### Example 1

### Rapid Thermocycling

A user loads 20 sample volumes comprising DNA samples, PCR reagents, and intercalating dye into a sample holder of a thermocycler. Each sample volume is 20 mL. A timing control system of the thermocycler controls the motion of the sample holder and four thermal zones mounted on rotating arms. The thermocycler begins thermally cycling the sample volumes. In each thermal cycle, the following occurs:

A) The sample holder moves down, and a first overshooting thermal zone held at a constant temperature of 135° C. moves horizontally in, such that the sample holder and the first overshooting thermal zone enter into thermal contact with each other. The sample volumes in the sample holder are heated to about 95° C. The sample holder moves up, and the first overshooting thermal zone moves horizontally out.

B) The sample holder moves down a second time, and a first target thermal zone held at a constant temperature of 95° C. moves horizontally in, such that the sample holder and the first target thermal zone enter into thermal contact with each other. The sample volumes in the sample holder are maintained at about 95° C. The sample holder moves up, and the first target thermal zone moves horizontally out.

C) The sample holder moves down a third time, and a second overshooting thermal zone held at a constant temperature of 8° C. moves horizontally in, such that the sample holder and the second overshooting thermal zone enter into thermal contact with each other. The sample volumes in the sample holder are cooled to about 55° C. The sample holder moves up, and the second overshooting thermal zone moves horizontally out.

D) The sample holder moves down a fourth time, and a second target thermal zone held at a constant temperature of 55° C. moves horizontally in, such that the sample holder and the second target thermal zone enter into thermal contact with each other. The sample volumes in the sample holder are maintained at about 55° C. The sample holder moves up, and the second target thermal zone moves horizontally out.

Conducting steps A through D completes one thermal cycle, which occurs in 2 seconds. 5 thermal cycles are conducted, and a detector of the thermocycler detects a fluorescent signal from the sample volumes indicative of nucleic acid amplification. The signals are transmitted to a computer and recorded as data. Additional thermal cycles are conducted, and fluorescent signal data is collected from each sample volume. This data is plotted as signal intensity versus thermal cycle number, displayed on a screen, and printed as a report.

### Example 2

### Metal Bath Constant Temperature Zones

A 200 microliter (μL) PCR sample tube is loaded with 50 μL of liquid solution comprising sample and PCR reagents at room temperature. Four metal bath thermal zones are configured as follows: a first overshooting thermal zone held constantly at 135° C., a first target thermal zone held constantly at 95° C., a second overshooting thermal zone held constantly at 8° C., and a second target thermal zone held constantly at 55° C. The PCR tube is moved in a cycle between the first overshooting thermal zone, the first target thermal zone, the second overshooting thermal zone, and the second target thermal zone. The PCR tube and its contents are heated and cooled between the target temperature levels of 95° C. and 55° C. at a rate of about 7° C./second.

### Example 3

### Operation from Motor Vehicle Power

A user takes a thermocycler apparatus into a motor vehicle. The user plugs the power supply of the thermocycler apparatus into the cigarette lighter power adaptor of the motor vehicle. The user loads samples and reagent into the thermocycler apparatus, which mix to form reaction mix. The user sets the thermocycler apparatus to run. The thermocycler apparatus draws power from the motor vehicle and cycles the temperature of the reaction mix. The samples are amplified and the results of the amplification are recorded.

### Example 4

### Remote Monitoring

A user loads samples and reagent into a thermocycler apparatus. The user initiates an amplification reaction with the thermocycler apparatus. The user travels to a separate location from the thermocycler apparatus. Live images of the progress of the amplification are viewed by the user on a remote monitoring device.

### Example 5

### Remote Control

A user loads samples and reagent into a thermocycler apparatus. The user initiates an amplification reaction with the thermocycler apparatus. The user travels to a separate location from the thermocycler apparatus. The user sends commands or instructions to the thermocycler apparatus from a remote device, instructing it to stop the amplification.

### Example 6

### Rapid Thermocycling of Single Tube with Two Zones

A user loads a single sample volume comprising DNA and/or RNA samples, PCR reagents, and intercalating dye into a sample holder of a thermocycler. A timing control system of the thermocycler controls the motion of the sample holder mounted on a swinging arm driven by a steering engine and two thermal zones driven by a stepper motor. The thermocycler begins thermally cycling the sample volume. In each thermal cycle, the following occurs:

A) A first cooling module and a second cooling module composing a cooling overshooting thermal zone held at a constant temperature of 8° C. move horizontally in opposite directions away from the sample holder to reach an open position, such that the sample volume and the cooling modules are not in thermal contact with each other.

B) The sample holder is swung with the swinging arm into a heating overshooting thermal zone and between a first heating module and a second heating module, wherein the first heating module and the second heating module are in an open position and the heating overshooting thermal zone is held at a constant temperature of 135° C.

C) The first heating module and the second heating module of the heating overshooting thermal zone move horizontally toward the sample holder to reach a closed position, so that the first heating module and the second heating module surround and in thermal contact with the sample volume. The sample volume is heated to about 95° C.

D) In some cases, if it is desired to hold the sample volume at a constant temperature of 95° C. for a length of time, the first heating module and the second heating module move horizontally away and toward the sample holder alternately to switch between the open position and the closed position, thereby being in thermal contact with the sample volume when the detected temperature is below about 95° C. and out of thermal contact with the sample volume when the detected temperature is above about 95° C.

E) The first heating module and the second heating module move horizontally in opposite directions away from the sample holder to reach an open position, such that the sample volume and the heating modules are not in thermal contact with each other.

F) The sample holder is swung with the swinging arm into the cooling overshooting thermal zone and between the first cooling module and the second cooling module, wherein the first cooling module and the second cooling module are in the open position.

G) The first cooling module and the second cooling module of the cooling overshooting thermal zone held at a constant temperature of 8° C. move horizontally toward the sample holder to reach a closed position, so that the first cooling module and the second cooling module surround and in thermal contact with the sample volume. The sample volume is cooled to about 55° C.

Conducting steps A) through G) completes one thermal cycle, which occurs in 2 seconds. 5 thermal cycles are conducted, and a detector of the thermocycler detects a fluorescent signal from the sample volume indicative of nucleic acid amplification. The signals are transmitted to a computer and recorded as data. Additional thermal cycles are conducted, and fluorescent signal data is collected from each sample volume. This data is plotted as signal intensity versus thermal cycle number, displayed on a screen, and printed as a report.

If it is desired to hold the sample volume at a constant temperature of 55° C. for a length of time after step G), then perform steps A) and B), followed by step H), wherein the first heating module and the second heating module move horizontally away and toward the sample holder alternately to switch between the open position and the closed position, thereby being in thermal contact with the sample volume when the detected temperature is below about 55° C. and out of thermal contact with the sample volume when the detected temperature is above about 55° C. Then, continue with step C) to heat the sample volume to about 95° C.

### Example 7

### Amplification and Detection of Hepatitis B Virus in Blood Samples

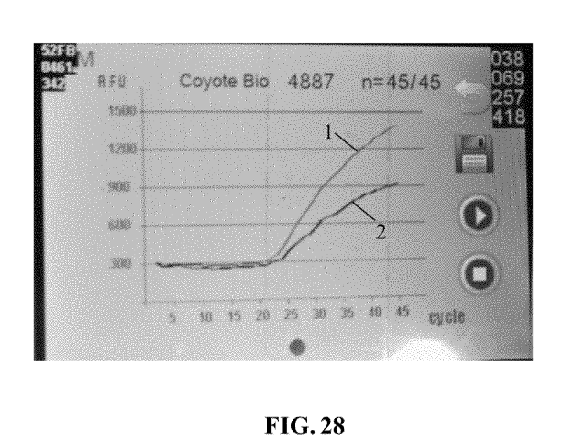

A PCR sample tube was loaded with a liquid solution containing a blood sample suspected of comprising HBV, and PCR reagents at room temperature. A thermal cycler of the present disclosure (e.g., a thermal cycler as described in Example 6) was used to perform the following amplification protocol: 1) heating the sample at a first overshooting temperature of about 115° C., until a first target temperature of about 94° C. is reached; 2) then, cooling the sample at a second overshooting temperature of about 20° C., until a second target temperature of about 48° C. is reached; and 3) repeating operations 1) and 2) for 45 cycles. As shown in FIG. 28, the target HBV was successfully detected.

### Example 8

### Amplification and Detection of Hepatitis C Virus (HCV)

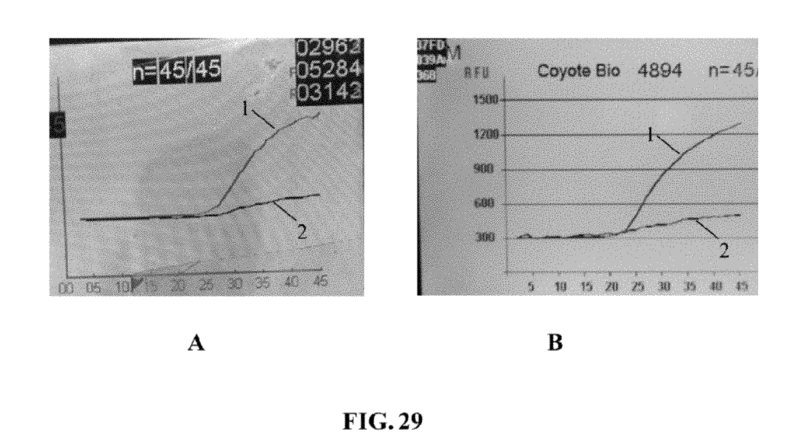

A PCR sample tube was loaded with a liquid solution containing a sample comprising HCV pseudovirus (FIG. 29 (panel A)) or a blood sample suspected of comprising HCV (FIG. 29 (panel B)), and PCR reagents at room temperature. A thermal cycler of the present disclosure (e.g., a thermal cycler as described in Example 6) was used to perform the following amplification protocol: 1) heating the sample at a first overshooting temperature of about 120° C., until a first target temperature of about 87° C. is reached; 2) then, cooling the sample at a second overshooting temperature of about 20° C., until a second target temperature of about 50° C. is reached; and 3) repeating operations 1) and 2) for 45 cycles. As shown in FIG. 29, the target HCV was successfully detected from both the HCV pseudovirus sample (FIG. 29 (panel A)) and the blood sample (FIG. 29 (panel B)).

### Example 9

### Detection of single nucleotide polymorphism (SNP) CYPC2C19

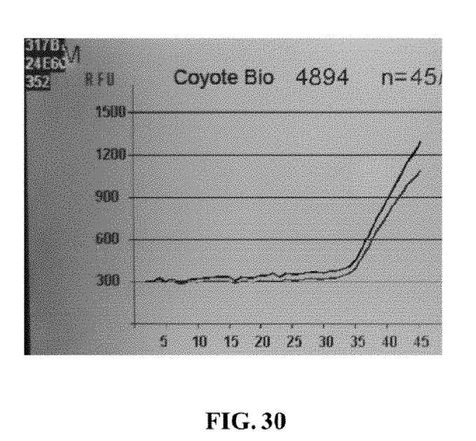

A PCR sample tube was loaded with a liquid solution containing a human blood sample suspected of comprising a nucleic acid molecule containing the SNP CYPC2C19, and PCR reagents at room temperature. A thermal cycler of the present disclosure (e.g., a thermal cycler as described in Example 6) was used to perform the following amplification protocol: 1) heating the sample at a first overshooting temperature of about 115° C., until a first target temperature of about 94° C. is reached; 2) then, cooling the sample at a second overshooting temperature of about 20° C., until a second target temperature of about 48° C. is reached; and 3) repeating operations 1) and 2) for 45 cycles. As shown in FIG. 30, the target SNP CYPC2C19 was successfully detected.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of conducting a chemical reaction in a sample contained in a sample holder, said reaction requiring cycling between at least two target temperature levels, comprising:
(a) placing said sample holder in thermal communication with a first overshooting thermal zone to achieve a first target temperature level;
(b) placing said sample holder in thermal communication with a second overshooting thermal zone to achieve a second target temperature level; and
(c) optionally repeating (a) and (b),
wherein said first overshooting thermal zone is at a higher temperature than said first target temperature level and said second overshooting thermal zone is at a lower temperature than said second target temperature level.

2. The method of claim 1, wherein (a) is performed with the aid of a first rotating arm capable of placing said first overshooting thermal zone in thermal communication with said sample holder, and/or wherein (b) is performed with the aid of a second rotating arm capable of placing said second overshooting thermal zone in thermal communication with said sample holder.

3. The method of claim 2, wherein said first overshooting thermal zone is mounted on said first rotating arm, and wherein said second overshooting thermal zone is mounted on said second rotating arm.

4. The method of claim 1, further comprising: in between (a) and (b), placing said sample holder in thermal communication with a first target thermal zone at said first target temperature level; and/or after (b), placing said sample holder in thermal communication with a second target thermal zone at said second target temperature level.

5. The method of claim 1, further comprising:
(d) in between (a) and (b), placing said sample holder in thermal communication with a first target thermal zone at said first target temperature level; and
(e) after (b), placing said sample holder in thermal communication with a second target thermal zone at said second target temperature level.

6. The method of claim 5, wherein said first overshooting thermal zone is at a temperature of about 110° C. to 140° C., said first target temperature level is about 87° C. to about 95° C., said second target temperature level is about 40° C. to about 70° C., and the second overshooting thermal zone is about 0° C. to about 30° C.

7. The method of claim 5, wherein one cycle of (a) through (e) is completed in less than or equal to about 2 seconds.

8. The method of claim 5, wherein (a) through (e) are repeated at least 5 times.

9. The method of claim 1, wherein said first overshooting thermal zone is at a temperature from about 110° C. to about 140° C.

10. (canceled)

11. The method of claim 1, wherein said second overshooting thermal zone is at a temperature from about 0° C. to about 30° C.

12. (canceled)

13. The method of claim 1, wherein said first target temperature level is from about 87° C. to about 95° C.

14. The method of claim 1, wherein said second target temperature level is from about 40° C. to about 70° C.

15. (canceled)

16. The method of claim 1, wherein said first overshooting thermal zone and said second overshooting thermal zone are powered by a 12 volt power supply.

17. The method of claim 1, wherein said sample holder is pre-loaded with amplification reagent prior to collecting said sample in said sample holder.

18. The method of claim 1, wherein said sample holder is placed in thermal communication with said first overshooting thermal zone and said second overshooting thermal zone using a first translational unit and a second translational unit, wherein said first translational unit subjects said first overshooting thermal zone and said second overshooting thermal zone to movement along a first plane, and wherein said second translational unit subjects said sample holder to movement along a second plane that is angled with respect to said first plane.

19. The method of claim 18, wherein (a) comprises using said first translational unit to move said first overshooting thermal zone to a first position and said second overshooting thermal zone to a second position along said first plane when said sample holder is raised away from said first plane, and subsequently using said second translational unit to lower said sample holder towards said first plane such that said sample holder is brought in thermal communication with said first overshooting thermal zone, and/or wherein (b) comprises using said first translational unit to move said second overshooting thermal zone to said first position and said first overshooting thermal zone to a third position when said sample holder is raised away from said first plane, and subsequently use said second translational unit to lower said sample holder towards said first plane such that said sample holder is brought in thermal communication with said second overshooting thermal zone.

20. (canceled)

21. The method of claim 18, wherein said first translational unit subjects said first overshooting thermal zone and said second overshooting thermal zone to simultaneous movement along said first plane.

22. The method of claim 18, wherein said movement along said second plane is towards or away from said first plane.

23. The method of claim 18, wherein said second plane is at an angle from about 45° to 90° with respect to said first plane.

24. The method of claim 1, wherein (a) is performed with the aid of a swinging arm capable of placing said sample holder in thermal communication with said first overshooting thermal zone, and/or wherein (b) is performed with the aid of said swinging arm capable of placing said sample holder in thermal communication with said second overshooting thermal zone.

25. The method of claim 24, wherein said sample holder is mounted on said swinging arm.

26.-84. (canceled)

85. An apparatus for conducting a reaction on a sample, comprising:
a sample holder that holds said sample during said reaction, wherein said reaction comprises cycling between at least two target temperature levels including a first target temperature level and a second target temperature level;
a first overshooting thermal zone and a second overshooting thermal zone, wherein said first overshooting thermal zone is at a higher temperature than said first target temperature level and said second overshooting thermal zone is at a lower temperature than said second target temperature level, or vice versa; and
a controller that is programmed to alternately and sequentially (i) place said sample holder in thermal communication with said first overshooting thermal zone to achieve said first target temperature level, and (ii) place said sample holder in thermal communication with said second overshooting thermal zone to achieve said second target temperature level.

86. The apparatus of claim 85, further comprising a first translational unit and a second translational unit, wherein said first translational unit subjects said first overshooting thermal zone and said second overshooting thermal zone to movement along a first plane, and wherein said second translational unit subjects said sample holder to movement along a second plane that is angled with respect to said first plane,
wherein said controller is operatively coupled to said first translational unit and said second translational unit, and
wherein said controller is programmed to subject said first overshooting thermal zone and said second overshooting thermal zone to movement along said first plane, and subject said sample holder to movement along said second plane, to alternately and sequentially (i) place said sample holder in thermal communication with said first overshooting thermal zone to achieve said first target temperature level, and (ii) place said sample holder in thermal communication with said second overshooting thermal zone to achieve said second target temperature level.

87. The apparatus of claim 86, wherein said first translational unit subjects said first overshooting thermal zone and said second overshooting thermal zone to simultaneous movement along said first plane.

88. (canceled)

89. The apparatus of claim 86, wherein said second plane is at an angle from about 45° to 90° with respect to said first plane.

90. The apparatus of claim 86, wherein said controller is programmed to:
(1) direct said first translational unit to move said first overshooting thermal zone to a first position and said second overshooting thermal zone to a second position along said first plane when said sample holder is raised away from said first plane;
(2) direct said second translational unit to lower said sample holder towards said first plane, thereby placing said sample holder in thermal communication with said first overshooting thermal zone to achieve said first target temperature level;
(3) direct said first translational unit to move said second overshooting thermal zone to said first position and said first overshooting thermal zone to a third position when said sample holder is raised away from said first plane; and
(4) direct said second translational unit to lower said sample holder towards said first plane, thereby placing said sample holder in thermal communication with said second overshooting thermal zone to achieve said second target temperature level.

91. (canceled)

92. (canceled)

93. The apparatus of claim 86, wherein said first translational unit and/or said second translational unit includes at least one motor or piezoelectric actuator.

94. The apparatus of claim 86, wherein said first translational unit and/or said second translational unit includes a track.

95.-99 (canceled)

100. The apparatus of claim 85, wherein said first overshooting thermal zone is a heating unit.

101. The apparatus of claim 85, wherein said second overshooting thermal zone is a cooling unit.
